(19) United States
(12) Patent Application Publication
(10) Pub. No.: US 2011/0207969 A1
(43) Pub. Date: Aug. 25, 2011

(54) PROCESS FOR MAKING POLYOL ETHERS

(76) Inventors: Michael M. Olken, Midland, MI (US); Michael L. Tulchinsky, Midland, MI (US)

(21) Appl. No.: 13/027,480

(22) Filed: Feb. 15, 2011

Related U.S. Application Data
(60) Provisional application No. 61/307,002, filed on Feb. 23, 2010.

(51) Int. Cl. C07C 41/05; B01J 21/18; B01J 37/02; B01J 37/16

(52) U.S. Cl. 568/679; 568/680; 502/185; 502/184

(57) ABSTRACT

The present invention generally relates to a process for making polyol ethers by reacting a polyol and a carbonyl compound together in the presence of hydrogen gas and a palladium hydrogenation catalyst on an acidic mesoporous carbon support.

### CROSS-REFERENCE TO RELATED APPLICATIONS

This application claims benefit from U.S. Provisional Patent Application No. 61/307,002, filed Feb. 23, 2010, the entire contents of which are hereby incorporated herein by reference.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC

Not applicable.

### BACKGROUND OF THE INVENTION

1. Field of the Invention

The present invention generally relates to a process for making polyol ethers by reacting a polyol and a carbonyl compound together in the presence of hydrogen gas and a palladium hydrogenation catalyst on an acidic mesoporous carbon support.

2. Description of the Related Art

Chemical and allied industries use polyol ethers such as, for example, glycerol ethers, glycol ethers and polyglycol ethers as, among other things, solvents, surfactants, wetting agents, emulsifying agents, lubricants, active ingredients in hard surface cleaning, laundry, cosmetics, personal care, ink formulations for ink-jet printing, as fabric softeners, preservatives, fragrance enhancers, and intermediates for the preparation of surfactants. They are also used in drug delivery applications, treatment of allergies, and as antimicrobial agents.

A wide variety of palladium catalysts and different catalytic activities thereof are known. The variety of the palladium catalysts and their catalytic activities are a function of, among other things, the following characteristics: methods of preparing such catalysts (e.g., impregnation or incipient wetness technique, ion exchange, deposition-adsorption, deposition-precipitation, or deposition-reduction); chemical composition and characteristics of the palladium starting material used in such preparation methods (e.g., H₂PdCl₄, NaPdCl₄, Pd(NO₃)₂, or (NH₃)₄Pd(NO₃)₂); whether or not anions residual from the palladium starting material remain in the palladium catalyst or are removed therefrom (e.g., by washing or during activation); use of a catalyst support or not; chemical composition of the catalyst support (e.g., a support comprising silicon dioxide, alumina, carbon, or zeolite); structural characteristics of the catalyst support (e.g., surface area, porosity, acidity, and particle shape); presence or absence of additional metal components; procedure, with respect to the palladium, by which the co-metal is added to the catalyst support (e.g., sequential to or simultaneously with the palladium); nature of the co-metal; amount of the co-metal relative to palladium; how the catalyst is activated, which in this context means how an ionic palladium species is reduced to its zero-valent active metallic form (e.g., hydrogen gas acting on a dry powder form at an elevated temperature or a solution phase activation); type of reaction for which the palladium catalyst is intended; and combinations of these differences, which combinations themselves produce yet more variability (e.g., palladium catalysts prepared by different methods may have different catalytic activities and selectivities in different reactions).

U.S. Pat. No. 5,446,208 mentions, among other things, a process for producing ether alcohols by hydrogenolysis of cyclic ketals in the presence of a palladium catalyst. The palladium catalyst can further comprise a co-metal such as ruthenium, rhodium, platinum, or nickel but such palladium bimetallic catalysts must have at least 50 weight percent (wt %) palladium and 50 wt % or less of the co-metal.

U.S. Pat. No. 5,446,210 mentions, among other things, a process for producing polyol ethers by reacting a mixture of at least one polyol and at least one carbonyl compound in the presence of a hydrogenation catalyst, and removing the polyol ether-containing reaction product from the hydrogenation catalyst. Examples indicate the process is not selective, producing on a molar basis significantly more alcohol by-product from reduction of the carbonyl compound than the polyol ether-containing compound.

Shi Y., et al., Straightforward selective synthesis of linear 1-O-alkyl glycerol and di-glycerol monoethers, Tetrahedron Letters, 2009; 50:6891-6893, mention, among other things, a process for producing 1-O-alkyl glycerol and di-glycerol monoethers generally employing an aldehyde, glycerol or di-glycerol, hydrogen gas, and a hydrogenation catalyst of palladium on carbon and a co-catalyst that is a strong Brønsted acid (i.e., a protic acid having an acid dissociation constant (pKa)≦2). The process requires the co-catalyst for achieving yields of the monoethers greater than trace yields. Shi Y., et al. mention a 40:1 ratio of glycerol or di-glycerol to aldehyde is optimal.

### BRIEF SUMMARY OF THE INVENTION

The present invention provides a process for making polyol ethers by reacting a polyol and a carbonyl compound in the presence of hydrogen gas and an improved palladium catalyst to give higher yields of the polyol ethers and increased selectivity of the polyol ethers over by-products.

In a first embodiment the present invention is a process for making a polyol ether, the process comprising contacting together under selective hydrogenating conditions an excess amount of a polyol, an amount of a carbonyl compound, an excess amount of hydrogen gas, and a catalytic amount of a palladium hydrogenation catalyst on an acidic mesoporous carbon support so as to provide the polyol ether (typically as a mixture of two or more polyol ethers such as, for example, a mixture comprising one, two or more different polyol monoethers and, optionally, one or more polyol diethers), wherein:

(a) the carbonyl compound is of formula (I):

R¹R²C═O (I)
wherein each of R¹ and R² independently is hydrogen atom (H), (C₁-C₅₀)alkyl, (C₂-C₅₀)alkenyl, (C₆-C₁₀)aryl-(C₁-C₅₀)alkyl, (C₆-C₁₀)aryl-(C₂-C₅₀)alkenyl-, or (C₃-C₁₂)cycloalkyl; or R¹ and R² together with the carbon atom to which they are both attached form a (C₃-C₁₂)cycloalkyl ring;

(b) the polyol is a compound of formula (II):

HO—[CH(R³)-Q-CR⁴(R⁵)—O]ₘ—H (II)
wherein m is an integer of from 1 to 2000;
each Q independently is a covalent bond (i.e., the -Q- is a covalent bond), L, X, L-X, X-L, or L-X-L, wherein each L independently is (C₁-C₁₄)alkylene, (C₁-C₁₄)heteroalkylene, or (C₂-C₁₄)alkenylene; and each X independently is (C₃-C₁₂)cycloalkylene, (C₂-C₁₂)heterocycloalkylene, (C₆-C₁₀)arylene, or (C₁-C₁₀)heteroarylene;
each of R³, R⁴, and R⁵ independently is H, (C₁-C₂₀)alkyl, (C₆-C₁₀)aryl-(C₁-C₁₀)alkyl, or (C₃-C₁₂)cycloalkyl; or R⁴ and R⁵ are together with the carbon atom to which they are both attached form a (C₃-C₁₂)cycloalkyl ring;

(c) the polyol ether comprises a compound of formula (IIIa), (IIIb), or (IIIc):

R¹R²C(H)—O—[CH(R³)-Q-CR⁴(R⁵)—O]ₘ—H (IIIa),

HO—[CH(R³)-Q-CR⁴(R⁵)—O]ₘ—CHR¹R² (IIIb), or

R¹R²C(H)—O—[CH(R³)-Q-CR⁴(R⁵)—O]ₘ—CHR¹R² (IIIc), or
a mixture of any two or more compounds of the formulas (IIIa), (IIIb), and (IIIc),

wherein m, Q, and R¹ to R⁵ are as defined previously; and each alkyl, alkylene, alkenyl, alkenylene, aryl, arylene, cycloalkyl, cycloalkylene, (C₁-C₁₄)heteroalkylene, and (C₂-C₁₂)heterocycloalkylene group independently is unsubstituted or substituted with from 1 to 10 substituent groups R^{S}, wherein each R^{S} is bonded to a carbon atom and independently is a hydroxyl (—OH), ═O, halo, di(C₁-C₂₀)alkylamino, (C₁-C₆)alkyl, —CHO (i.e., —C(═O)—H), —CO(C₁-C₆)alkyl (i.e., —C(═O)—(C₁-C₆)alkyl), —CO₂H, —CO₂(C₁-C₆)alkyl, —CON((C₁-C₆)alkyl)₂, (C₁-C₆)alkoxy, (C₂-C₆)alkynyl, or —SH;

(d) the palladium hydrogenation catalyst comprises palladium(0) or a palladium(0)-(co-metal) comprising palladium(0) in the presence of at least one co-metal, wherein the co-metal is lanthanum, yttrium, nickel, zinc, copper, manganese, cobalt, iron, chromium, vanadium, titanium, scandium, or a lanthanoid other than lanthanum (believe, without being bound by theory, that the co-metal is not zero valent but ionic); the palladium(0) or palladium(0)-(co-metal) being supported on a surface of the acidic mesoporous carbon support; the palladium hydrogenation catalyst having been prepared by impregnation or deposition-adsorption of a PdCl₂ (e.g., H₂PdCl₄) or independently a PdCl₂ and a corresponding co-metal chloride, respectively, on and into the acidic mesoporous carbon support so as to give an impregnated or deposited-adsorbed material, followed by an activating reduction of the palladium of the impregnated or deposited-adsorbed material so as to produce the palladium hydrogenation catalyst;

(e) the acidic mesoporous carbon support is characterizable as having a percent mesoporosity of greater than 15%, wherein percent mesoporosity is equal to 100 times mesopore surface area (square meters per gram (m²/g)) of the acidic mesoporous carbon support divided by Brunauer-Emmett-Teller (BET) surface area (square meters per gram) of the acidic mesoporous carbon support;

(f) the excess amount of the polyol is relative to the amount of the carbonyl compound and is characterizable by a molar ratio of the polyol to the carbonyl compound that is greater than or equal to 3 to 1 (≧3:1); and

(g) the process produces the polyol ether in at least 30 percent yield (i.e., sum of percent yields of all polyol ethers produced is at least 30%) based on the amount of the carbonyl compound and the process is characterizable by a molar selectivity ratio of greater than 10:1 for producing the polyol ether over a potential alcohol by-product of formula (IV) R¹R²CHOH (IV), wherein R¹ and R² are as defined previously (i.e., (sum of moles of polyol ethers produced) divided by the moles of alcohol by-product of formula (IV) produced, if any, is greater than 10:1).

In a second embodiment the present invention is a process for preparing a palladium hydrogenation catalyst, the process comprising impregnating or depositing-adsorbing a PdCl₂ or independently a PdCl₂ and a corresponding co-metal chloride that is lanthanum chloride, yttrium chloride, nickel chloride, zinc chloride, copper chloride, manganese chloride, cobalt chloride, iron chloride, chromium chloride, vanadium chloride, titanium chloride, scandium chloride, or a lanthanoid chloride other than lanthanum chloride on and in an acidic mesoporous carbon support to give an impregnated or deposited-adsorbed material; and activatingly reducing the palladium of the impregnated or deposited-adsorbed material so as to produce a palladium hydrogenation catalyst comprising palladium(0) or a palladium(0)-(co-metal) comprising palladium(0) in the presence of at least one co-metal (believed co-metal is not zero valent but ionic), wherein the least one co-metal is lanthanum, yttrium, nickel, zinc, copper, manganese, cobalt, iron, chromium, vanadium, titanium, scandium, or a lanthanoid other than lanthanum, the palladium(0) or palladium(0)-(co-metal) being supported on a surface of the acidic mesoporous carbon support, wherein the acidic mesoporous carbon support is characterizable as having a percent mesoporosity of greater than 15%, wherein percent mesoporosity is equal to 100 times mesopore surface area (m²/g) of the acidic mesoporous carbon support divided by Brunauer-Emmett-Teller (BET) surface area (m²/g) of the acidic mesoporous carbon support; and when a co-metal chloride is employed the depositing-adsorbing steps can be performed sequentially or essentially simultaneously and the activatingly reducing steps can be performed sequentially or essentially simultaneously (e.g., the activatingly reducing of a palladium chloride-containing deposited-adsorbed material can be performed before or after depositing-adsorbing the co-metal chloride and also before or after activatingly reducing a co-metal chloride-containing deposited-adsorbed material).

In a third embodiment the present invention is the palladium hydrogenation catalyst prepared in the second embodiment.

As used herein, the term “acidic mesoporous carbon support” means a finely divided substance consisting essentially of a matrix of carbon atoms wherein at least some of the carbon atoms of the carbon atom matrix are covalently bonded to acidic functional groups (e.g., —CO₂H), the substance being characterizable by a percent mesoporosity as defined previously and measured as described later.

The terms “activating reduction” and “activatingly reducing” mean adding electrons or hydrogen (e.g., via hydrogen gas or a hydride reagent such as, for example, sodium borohydride) so as to produce a functional catalyst.

The terms “Brunauer-Emmett-Teller surface area” and “mesopore surface area” are described later by respective procedures used to measure the surface areas.

The term “catalytic amount” means a molar amount that is less than a molar amount of the carbonyl compound and at least a minimum quantity that is sufficient to facilitate production of the at least 30% yield of the polyol ether within 24 hours reaction time.

The term “deposition-adsorption” means the accumulation of material (e.g., a metal complex such as a metal salt) onto a surface of a carrier when the material is slurried in an aqueous solution. The resulting adsorbed material would be retained by the carrier even after washing the carrier with deionized or distilled water. A detailed description of a preferred deposition-adsorption technique is provided later.

The term “excess hydrogen gas” means number of moles of a gaseous substance having a molecular formula H₂ greater than number of moles of the carbonyl compound.

The term “hydrogenation” means a reaction of hydrogen with reduction in which an element (e.g., oxygen, nitrogen, sulfur, carbon, or halogen) is withdrawn from, hydrogen is added to, or the element is withdrawn from and hydrogen is added to, a molecule. Examples of hydrogenation are addition of hydrogen to a reactive molecule (e.g., addition of hydrogen to H₂PdCl₄ to give Pd(0) and 4HCl) and incorporation of hydrogen accompanied by cleavage of the molecule (i.e., hydrogenolysis, e.g., reductive cleavage of an acetal or ketal to a monoether).

The term “impregnation” means permeate with a wetted, melted, or molten substance substantially throughout (e.g., via an incipient wetness technique), preferably to a point where essentially all of a liquid phase substance is adsorbed, producing a liquid-saturated but unagglomerated solid. A detailed description of a preferred impregnation technique is provided later.

The term “lanthanoid” means an element having an atomic number of from 57 to 71 of the Periodic Table of the Elements. For example lanthanum is a lanthanoid having the atomic number 57. Unless otherwise noted, the phrase “Periodic Table of the Elements” refers to the official periodic table, version dated Jun. 22, 2007, published by the International Union of Pure and Applied Chemistry (IUPAC). Also any references to a Group or Groups shall be to the Group or Groups reflected in this Periodic Table of the Elements.

The term “molar ratio” means a unitless rational or irrational number calculated by dividing number of moles of a first compound by number of moles of a second compound.

The term “palladium-(co-metal) co-adsorption” means a substance comprising palladium and the at least one co-metal, the substance being formed by a process comprising accumulation of palladium and accumulation of the at least one co-metal on a surface of the acidic mesoporous carbon support, wherein such accumulations can occur essentially simultaneously, sequentially, or a combination thereof.

The term “palladium hydrogenation catalyst” means a substance comprising palladium(0) that is effective for increasing rate of reaction of hydrogen with a carbonyl-containing compound or an intermediate derivative thereof (e.g., an acetal or ketal derivative thereof formed in situ) to produce an ether-containing compound.

The term “percent yield” means a number of parts of the polyol ether produced per 100 parts of the carbonyl compound employed.

The term “polyol” means an organic compound having at least two hydroxyl groups, each bonded to a different carbon atom.

The term “polyol ether” means an organic compound having at least one ether functional group, or a mixture of two or more such organic compounds (typically the polyol ether produced in the process of the first embodiment is a mixture of two or more polyol ethers such as, for example, a mixture comprising one, two or more different polyol monoethers and, optionally, one or more polyol diethers).

The term “selective hydrogenating conditions” mean reaction conditions such as environmental parameters and other reaction features under which a hydrogenation reaction is conducted that yields the polyol ether as described previously. The environmental parameters and other features are described in detail later.

The invention process advantageously produces the polyol ether without a need for any added acid co-catalyst or other additives. Acid co-catalyst additives are known to undesirably cause condensation between two carbonyl compounds, polycondensation of polyols, deactivate certain hydrogenation catalysts, or a combination thereof in prior art (i.e., non invention) processes. The invention process also advantageously produces the polyol ether in high yields (typically greater than 70% yield) and selectivities over by-products. The invention discovered that the acidic mesoporous carbon support having a minimum percent mesoporosity or greater facilitates increased catalytic activity of the palladium hydrogenation catalyst compared to other palladium catalysts on carbon that lack the acidity and percent mesoporosity features thereof and have a same palladium composition weight percent, and even compared to non-invention palladium catalysts on mesoporous carbon that have been prepared by methods other than the instant impregnation or deposition-adsorption, such other methods being, for example, co-precipitation or calcination (e.g., to form alloys).

The higher yields of and increased selectivities for the polyol ethers makes the invention process especially valuable in the preparation of polyol ethers for use as, for example, solvents, surfactants, wetting agents, emulsifying agents, lubricants, active ingredients in hard surface cleaning, laundry, cosmetics, personal care, ink formulations for ink-jet printing, as fabric softeners, preservatives, fragrance enhancers, and intermediates for the preparation of surfactants. They are also used in drug delivery applications, treatment of allergies, and as antimicrobial agents.

Additional embodiments are described in the accompanying drawings and the remainder of the specification, including the claims.

### BRIEF DESCRIPTION OF THE DRAWING(S)

Some embodiments of the present invention are described herein in relation to the accompanying drawing(s), which will at least assist in illustrating various features of the embodiments.

FIG. 1 is gas chromatography (GC) based area percent values of glycerol monoethers (3-pentyloxy-1,2-propanediol+2-pentyloxy-1,3-propanediol) versus time for each of five successive reaction runs of Example S reusing a same batch of invention catalyst in each successive reaction run.

FIG. 2 is GC area percent of by-product 1-pentanol selectivity in reductive etherification with catalyst reuse from the five successive reaction runs of Example S.

### DETAILED DESCRIPTION OF THE INVENTION

The present invention provides a process for making polyol ethers by reacting a polyol and a carbonyl compound in the presence of hydrogen gas and a palladium hydrogenation catalyst on an acidic mesoporous carbon support to give in higher yields of the polyol ethers and increased selectivity of the polyol ethers over by-products, all as summarized previously.

For purposes of United States patent practice and other patent practices allowing incorporation of subject matter by reference, the entire contents—unless otherwise indicated—of each U.S. patent, U.S. patent application, U.S. patent application publication, PCT international patent application and WO publication equivalent thereof, referenced in the instant Summary or Detailed Description of the Invention are hereby incorporated by reference. In an event where there is a conflict between what is written in the present specification and what is written in a patent, patent application, or patent application publication, or a portion thereof that is incorporated by reference, what is written in the present specification controls.

In the present application, any lower limit of a range of numbers, or any preferred lower limit of the range, may be combined with any upper limit of the range, or any preferred upper limit of the range, to define a preferred aspect or embodiment of the range. Each range of numbers includes all numbers, both rational and irrational numbers, subsumed within that range (e.g., the range from about 1 to about 5 includes, for example, 1, 1.5, 2, 2.75, 3, 3.80, 4, and 5).

In an event where there is a conflict between a unit value that is recited without parentheses, e.g., 2 inches, and a corresponding unit value that is parenthetically recited, e.g., (5 centimeters), the unit value recited without parentheses controls.

In the event there is a discrepancy between a chemical name and structure, the structure controls.

As used herein, “a,” “an,” “the,” “at least one,” and “one or more” are used interchangeably. In any aspect or embodiment of the instant invention described herein, the term “about” in a phrase referring to a numerical value may be deleted from the phrase to give another aspect or embodiment of the instant invention. In the former aspects or embodiments employing the term “about,” meaning of “about” can be construed from context of its use. Preferably “about” means from 90 percent to 100 percent of the numerical value, from 100 percent to 110 percent of the numerical value, or from 90 percent to 110 percent of the numerical value. In any aspect or embodiment of the instant invention described herein, the open-ended terms “comprising,” “comprises,” and the like (which are synonymous with “including,” “having,” and “characterized by”) may be replaced by the respective partially closed phrases “consisting essentially of,” consists essentially of,” and the like or the respective closed phrases “consisting of,” “consists of,” and the like to give another aspect or embodiment of the instant invention. In the present application, when referring to a preceding list of elements (e.g., ingredients), the phrases “mixture thereof,” “combination thereof,” and the like mean any two or more, including all, of the listed elements. The term “or” used in a listing of members, unless stated otherwise, refers to the listed members individually as well as in any combination, and supports additional embodiments reciting any one of the individual members (e.g., in an embodiment reciting the phrase “10 percent or more,” the “or” supports another embodiment reciting “10 percent” and still another embodiment reciting “more than 10 percent.”). The term “optionally” means “with or without.” For example, “optionally an additive” means with or without an additive. The term “plurality” means two or more, wherein each plurality is independently selected unless indicated otherwise. The symbols “≦” and “≧” respectively mean less than or equal to and greater than or equal to. The symbols “<” and “>” respectively mean less than and greater than.

As used herein, the terms “(C₁-C₅₀)alkyl” and “(C₁-C₂₀)alkyl” mean a straight or branched saturated hydrocarbon radical of from 1 to 50 carbon atoms or from 1 to 20 carbon atoms respectively (e.g., methyl, ethyl, 1-propyl, 2-propyl, 1-butyl, 2-butyl, 1,1-dimethylethyl, et cetera. The alkyl groups can be unsubstituted or substituted as described previously.

The term “(C₂-C₅₀)alkenyl” means a straight or branched, unsaturated non-aromatic hydrocarbon radical of from 2 to 50 carbon atoms and 1, 2, or 3 carbon-carbon double bonds. The alkenyl group can be unsubstituted or substituted as described previously.

The term “(C₁-C₁₄)alkylene” means a straight or branched saturated hydrocarbon diradical of from 1 to 14 carbon atoms. The alkylene group can be unsubstituted or substituted as described previously.

The term “(C₂-C₁₄)alkenylene” means a straight or branched, unsaturated non-aromatic hydrocarbon diradical of from 2 to 14 carbon atoms and 1, 2, or 3 carbon-carbon double bonds. The alkenylene group can be unsubstituted or substituted as described previously.

The term “(C₆-C₁₀)aryl” means an aromatic monocyclic or bicyclic hydrocarbon radical of from 6 to 10 ring atoms (e.g., phenyl or naphthyl). The aryl group can be unsubstituted or substituted as described previously.

The term “(C₆-C₁₀)arylene” means an aromatic monocyclic or bicyclic hydrocarbon diradical of from 6 to 10 ring atoms (e.g., phenylene or naphthylene). The arylene group can be unsubstituted or substituted as described previously.

The terms “(C₆-C₁₀)aryl-(C₁-C₅₀)alkyl” and “(C₆-C₁₀)aryl-(C₂-C₅₀)alkenyl” mean a (C₆-C₁₀)aryl substituted (C₁-C₅₀)alkyl or (C₂-C₅₀)alkenyl, wherein the (C₆-C₁₀)aryl, (C₁-C₅₀)alkyl, and (C₂-C₅₀)alkenyl are as described previously.

The term (C₃-C₁₂)cycloalkyl” means a non-aromatic monocyclic hydrocarbon radical of from 3 to 12 ring atoms and saturated (i.e., 0 carbon-carbon double bonds) or unsaturated (i.e., 1 or 2 carbon-carbon double bonds Examples of saturated (C₃-C₁₂)cycloalkyl are cyclopropyl, cyclobutyl, cyclopentyl, cyclohexyl, et cetera to cyclododecyl. Examples of unsaturated (C₃-C₁₂)cycloalkyl are cyclopropen-1-yl, cyclobuten-3-yl, and cyclopentadien-5-yl. The cycloalkyl group can be unsubstituted or substituted as described previously.

The term “(C₃-C₁₂)cycloalkylene” means a non-aromatic monocyclic hydrocarbon diradical of from 3 to 12 ring atoms and saturated (i.e., 0 carbon-carbon double bonds) or unsaturated (i.e., 1 or 2 carbon-carbon double bonds). Examples of saturated (C₃-C₁₂)cycloalkylene are cyclopropylene, cyclobutylene, cyclopentylene, cyclohexylene, et cetera to cyclododecylene, Examples of unsaturated (C₃-C₁₂)cycloalkylene are cyclopropen-1,3-diyl and cyclopentadien-1,2-diyl. The cycloalkylene group can be unsubstituted or substituted as described previously.

The term “(C₁-C₁₄)heteroalkylene” means a straight or branched, non-aromatic heterohydrocarbon diradical of from 1 to 14 carbon atoms; and saturated (i.e., 0 carbon-carbon double bonds) or unsaturated (i.e., 1, 2, or 3 carbon-carbon double bonds; and 1 to 4 heteroatoms, each heteroatom independently being O, S, N, or P. Examples are CH₂CH₂O, CH₂CH₂CH₂O, CH₂CH₂CH₂CH₂O, N(H)CH₂CH₂N(H), CH₂CH₂SCH₂, and PCH₂. The heteroalkylene group can be unsubstituted or substituted as described previously.

The term “(C₂-C₁₂)heterocycloalkylene” means a non-aromatic monocyclic heterohydrocarbon diradical of from 2 to 14 carbon atoms; and saturated (i.e., 0 carbon-carbon double bonds) or unsaturated (i.e., 1, 2, or 3 carbon-carbon double bonds; and 1 to 4 heteroatoms, each heteroatom independently being O, S, N, or P. Examples are and epoxide-2-yl and tetrahydrofuran-2-yl. The heterocycloalkylene group can be unsubstituted or substituted as described previously.

The term “(C₁-C₁₀)heteroarylene” means an aromatic monocyclic or bicyclic heterohydrocarbon diradical of from 1 to 10 carbon atoms; and 1 to 4 heteroatoms, each heteroatom independently being O, S, N, or P. Examples are tetrazol-1,5-diyl and pyridine-2,5-diyl. The heteroarylene group can be unsubstituted or substituted as described previously.

In some embodiments the carbonyl compound is the compound of formula (I) wherein R¹ is H and R² is (C₁-C₅₀)alkyl, (C₂-C₅₀)alkenyl, (C₆-C₁₀)aryl-(C₁-C₅₀)alkyl, (C₆-C₁₀)aryl-(C₂-C₅₀)alkenyl-, or (C₃-C₁₂)cycloalkyl. In some embodiments each of R¹ and R² independently is (C₁-C₅₀)alkyl, (C₂-C₅₀)alkenyl, (C₆-C₁₀)aryl-(C₁-C₅₀)alkyl, (C₆-C₁₀)aryl-(C₂-C₅₀)alkenyl-, or (C₃-C₁₂)cycloalkyl. In some embodiments R¹ and R² together with the carbon atom to which they are both attached form a (C₃-C₁₂)cycloalkyl ring. In some embodiments R¹ or R² but not both, or each of R¹ and R², independently is (C₁-C₅₀)alkyl, in other embodiments (C₂-C₅₀)alkenyl, in still other embodiments (C₆-C₁₀)aryl-(C₁-C₅₀)alkyl, in even other embodiments (C₆-C₁₀)aryl-(C₂-C₅₀)alkenyl-, and in yet other embodiments (C₃-C₁₂)cycloalkyl. Preferably R¹ and R² are not both H, i.e., the carbonyl compound is not formaldehyde.

In some embodiments the carbonyl compound is glutaraldehyde, formaldehyde, acetaldehyde, acrolein, propionaldehyde, butyraldehyde, crotonaldehyde, caproic aldehyde, caprylic aldehyde, capric aldehyde, lauryl aldehyde, myristyl aldehyde, cetyl aldehyde, stearyl aldehyde, oley aldehyde, elaidyl aldehyde, linolyl aldehyde, linolenyl aldehyde, behenyl aldehyde, erucyl aldehyde, isobutyraldehyde, n-butyraldehyde, methylethylketone, 2-undecanone, normal-decanal (i.e., n-decanal), 2-methylundecanal, n-valeraldehyde, iso-valeraldehyde, 2-methylbutanal, n-hexanal, n-heptanal, 2-ethylhexanal, acetone, methylethylketone, 2-pentanone, 3-pentanone, cinnamaldehyde, levulinic acid, 1,3-cyclohexanedicarboxaldehyde, 1,4-cyclohexanedicarboxaldehyde, cyclohexanone, or a mixture of two or more thereof.

A more preferred carbonyl compound is n-butyraldehyde, methylethylketone, 2-undecanone, n-decanal, 2-methylundecanal, n-valeraldehyde, iso-valeraldehyde, 2-methylbutanal, n-hexanal, n-heptanal, 2-ethylhexanal, acetone, methylethylketone, 2-pentanone, 3-pentanone, cinnamaldehyde, levulinic acid, 1,3-cyclohexanedicarboxaldehyde, 1,4-cyclohexanedicarboxaldehyde, cyclohexanone, or a mixture of two or more thereof. In one particular embodiment, the carbonyl compound is a mixture of 1,3-cyclohexanedicarboxaldehyde and 1,4-cyclohexanedicarboxaldehyde. In the case of unsaturated carbonyl compounds and, less likely, aromatic compounds (e.g., phenyl-containing compounds), the double bonds thereof may be hydrogenated during the reaction to form saturated derivatives thereof (e.g., saturated carbonyl compounds or, less likely, cyclohexyl-containing compounds, respectively).

As mentioned previously, the invention encompasses carbonyl compounds that are unsubstituted or substituted. The substituted carbonyl compounds are capable of undergoing additional or tandem reactions to form further materials during the process of the invention. For instance, where a substituent R^{S} is carboxylic acid (—COOH), such as in levulinic acid, the carboxylic acid moiety is capable of undergoing esterification in tandem with the etherification of the carbonyl portion of the molecule.

The carbonyl compounds are available from a variety of commercial sources, can be readily prepared by a person of ordinary skill in the art using well known techniques, or both. The source of the carbonyl compound and its method of preparation are not critical to the invention. For instance, aldehydes derived from seed oils or other natural sources are encompassed, as well as aldehydes that are by-products of industrial processes, or aldehydes derived from hydroformylation reactions.

In some embodiments the polyol is the compound of formula (II) wherein m is 1 and -Q- is methylene or —CH(OH)—. Preferably R³ to R⁵ each are H, i.e., the compound of formula (II) is glycerol.

In some embodiments the polyol is the compound of formula (II) wherein m is 1 and -Q- is a covalent bond, thereby giving a polyol that is compound of formula (II-a):

HO—CH(R³)—CR⁴(R⁵)—OH (II-a). In some embodiments R³ and R⁴ each are H and R⁵ is (C₁-C₁₄)alkyl. Preferably the (C₁-C₁₄)alkyl is (C₁-C₆)alkyl. More preferably R³ and R⁴ each are H and R⁵ is methyl, i.e., the compound of formula (II-a) is propylene glycol.

In other embodiments m is 2 or more, thereby giving a polyol that is compound of formula (II-b):

HO—CH(R³)-Q-CR⁴(R⁵)—O—[CH(R³)-Q-CR⁴(R⁵)—O]ₘ₋₁—H (II-b). When m is 2 or more each Q can be the same or different. Preferably, however, each Q is the same. Preferably each Q independently is (C₁-C₁₄)alkylene or (C₁-C₁₄)heteroalkylene, more preferably each Q independently is a (C₁-C₆)alkylene, and still more preferably each Q independently is methylene (i.e., CH₂) or ethylene (CH₂CH₂). Preferably m is 500 or less, more preferably 100 or less, still more preferably 10 or less, and even more preferably 5 or less. In some embodiments m is 2, in other embodiments m is 3, in still other embodiments m is 4, in yet other embodiments m is 5, and in still yet other embodiments m is 6.

In some embodiments the polyol is a compound of formula (II) wherein each of R³ to R⁵ is H. In some embodiments two of R³ to R⁵ is H and the other one of R³ to R⁵ is (C₁-C₂₀)alkyl, (C₆-C₁₀)aryl-(C₁-C₁₀)alkyl, or (C₃-C₁₂)cycloalkyl. In some embodiments the other one of R³ to R⁵ is (C₁-C₂₀)alkyl, in other embodiments (C₆-C₁₀)aryl-(C₁-C₁₀)alkyl, and in still other embodiments (C₃-C₁₂)cyclo alkyl.

In some embodiments one of R³ to R⁵ is H and the other two of R³ to R⁵ each independently is (C₁-C₂₀)alkyl, (C₆-C₁₀)aryl-(C₁-C₁₀)alkyl, or (C₃-C₁₂)cycloalkyl. In some embodiments each of the other two of R³ to R⁵ independently is (C₁-C₂₀)alkyl. In some embodiments R³ is H and R⁴ and R⁵ are together with the carbon atom to which they are both attached form a (C₃-C₁₂)cycloalkyl ring.

In some embodiments the polyol comprises a 1,2-diol moiety [i.e., C(OH)—C(OH)]. In some embodiments the polyol comprises a 1,3-diol moiety [i.e., C(OH)—C—C(OH)]. In some embodiments the polyol lacks a 1,2-diol and 1,3-diol moiety.

In some embodiments the polyol is a polyalkylene glycol, more preferably a polyethylene glycol, polypropylene glycol, or polybutylene glycol. In some embodiments the polyol is ethylene glycol; diethylene glycol; triethylene glycol; tetraethylene glycol; a polyethylene glycol with a number average molecular weight ranging from 62 grams per mole (g/mol) to 620 g/mol); 1,2-propylene glycol; 1,3-propylene glycol; 1,2-butylene glycol; 1,3-butylene glycol; 1,4-butylene glycol; or a mixture of any two or more thereof.

In some embodiments the polyol is glycerol; sorbitol; mannitol; 2-hydroxymethyl-1,3-propanediol; 1,1,1-tris(hydroxymethyl)ethane; trimethylolpropane; pentaerythritol; diglycerol; or a mixture of any two or more thereof. Examples of such polyols are diglycerol isomers drawn below:

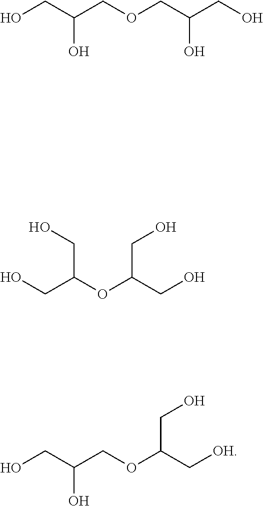

Preferably the at least two hydroxyl groups of the polyol are non-phenolic or non-enolic, and more preferably both.

The polyol is available from a variety of commercial sources, can be readily prepared by a person of ordinary skill in the art using well known techniques, or both. The source of the polyol is not critical to the invention. In some embodiments, obtaining the polyol from renewable non-petroleum sources such as a biobased feedstock is desirable. Bio-based polyols are described, for instance, in U.S. Patent Application Publication numbers US 2007/0129451 A1 and US 2008/0103340 A1.

In some embodiments the polyol ether comprises the compound of formula (IIIa), in other embodiments the compound of formula (IIIb), in still other embodiments the compound of formula (IIIc), and in yet other embodiments the mixture of any two or more compounds of the formulas (IIIa), (IIIb), and (IIIc). Purity and structure or composition of the polyol ether can be readily determined using structure or composition information about the carbonyl compound and polyol and well known characterization techniques. Examples of suitable well known characterization techniques are chromatography (e.g., gas chromatography (GC)), nuclear magnetic resonance (NMR) spectroscopy (e.g., proton NMR, carbon-13 NMR, or both), mass spectrometry (MS; e.g., GC-MS), infrared spectroscopy, one or more polymer characterization techniques (e.g., dynamic mechanical analysis (DMA), differential scanning calorimetry (DSC), and thermogravimetric analysis (TGA)), or a combination thereof.

In some embodiments the invention process forms in situ an acyclic acetal or ketal intermediate from the polyol and carbonyl compound. Preferably the invention process forms the acyclic acetal or ketal with a polyol lacking the aforementioned 1,2-diol and 1,3-diol moieties. In some embodiments the invention process forms in situ a cyclic acetal or ketal intermediate. Preferably the invention process forms the cyclic acetal or ketal with a polyol comprising the aforementioned 1,2-diol or 1,3-diol moiety. The invention contemplates recycling unreacted acetal or ketal intermediate, with or without isolation thereof from an invention reaction mixture containing the acetal or ketal intermediate.

The structure or composition information about the carbonyl compound and polyol is readily ascertained and helpful for determining the polyol ether compound of formula (III) prepared therefrom. For example, the R¹ and R² are known from the carbonyl compound and Q, m, and R³ to R⁵ are known from the polyol, and so the Q, m, and R¹ to R⁵ of the compound of formula (III) will be the same, respectively. Thus, R¹ and R² of preferred compounds of formula (III) are the same as the aforementioned preferred R¹ and R² of the carbonyl compound of formula (I) and Q, m, and R³ to R⁵ of the preferred compounds of formula (III) are the same as the aforementioned preferred Q, m, and R³ to R⁵ of the polyol of formula (II).

The palladium hydrogenation catalyst useful in the present invention can be characterized by its intrinsic acidity. Preferably a 5 wt % suspension of the palladium hydrogenation catalyst has a potential of hydrogen (pH) of less than or equal to pH 6 (e.g., from pH 2 to pH 6), in some embodiments from pH 3 to pH 5, and more preferably from pH 3.5 to pH 4.5 (e.g., about pH 4).

The palladium hydrogenation catalyst useful in the present invention can be characterized by a weight percent of palladium, a weight percent of any co-metal, or both, all based on total weight of the palladium hydrogenation catalyst, which characterization is referred to herein as catalyst composition. The invention contemplates any catalyst composition. Preferably the catalyst composition is from 0.01 wt % to 30 wt % of palladium based on total weight of the palladium hydrogenation catalyst (that is from 0.01 g to 30 g palladium per 100 g or the catalyst). More preferably the catalyst composition is from 0.1 wt % to 10 wt % palladium, and still more preferably 5 wt % palladium or less. Still more preferably the catalyst composition is 1 wt % palladium or more, and even more preferably 2 wt % palladium or more. When the palladium hydrogenation catalyst further comprises a co-metal, preferably the palladium hydrogenation catalyst comprises from 0.01 wt % to 20 wt % of the co-metal based on total weight of the palladium hydrogenation catalyst. More preferably the palladium hydrogenation catalyst comprises from 0.1 wt % to 10 wt % of the co-metal, and still more preferably 1 wt % co-metal or more, and even more preferably 2 wt % co-metal or more. In some embodiments the catalyst composition amounts of the palladium and co-metal, if any, independently are as described later in any one of the Examples.

When a co-metal is employed in the palladium hydrogenation catalyst useful in the present invention, the palladium hydrogenation catalyst can be characterized by a ratio of weight palladium to weight of the co-metal, which characterization is referred to herein as catalyst metal weight/weight ratio. The invention contemplates any catalyst metal weight/weight ratio. The catalyst metal weight/weight ratio can be conveniently calculated from the catalyst composition values. For example, a catalyst composition of 20 wt % palladium and 5 wt % co-metal gives a catalyst metal weight/weight ratio of 80:20 (i.e., 4:1). Preferably the catalyst metal weight/weight ratio is from 80 palladium:20 co-metal to 20 palladium:80 co-metal.

The palladium hydrogenation catalyst useful in the present invention can be characterized by a weight percent of catalyst per unit weight of the carbonyl compound, which characterization is referred to herein as catalyst loading. The invention contemplates any catalyst loading. Preferably the catalyst loading is from 0.1 wt % to 50 wt % of the palladium hydrogenation catalyst based on weight of the carbonyl compound (that is from 0.1 g to 50 g catalyst per 100 g or the carbonyl compound). More preferably the catalyst loading is from 1 wt % to 20 wt %, and still more preferably 10 wt % or less. Still more preferably the catalyst loading is 2 wt % or more, and even more preferably 2.5 wt % or more. In some embodiments the catalyst loading is as described later in any one of the Examples.

The palladium hydrogenation catalyst useful in the present invention can be characterized by absence of, or presence and identity of, the co-metal. In some embodiments the palladium hydrogenation catalyst lacks a co-metal. In some embodiments the palladium hydrogenation catalyst contains one co-metal. In some embodiments the co-metal is lanthanum, nickel, zinc, copper, manganese, cobalt, iron, chromium, vanadium, titanium, or scandium. In some embodiments the co-metal is lanthanum or nickel. In some embodiments the co-metal is lanthanum. In some embodiments the co-metal is nickel. In some embodiments the co-metal is zinc, copper, manganese, cobalt, iron, chromium, vanadium, titanium, or scandium. In some embodiments the co-metal is zinc. In some embodiments the co-metal is copper. In some embodiments the co-metal is manganese. In some embodiments the co-metal is cobalt. In some embodiments the co-metal is iron. In some embodiments the co-metal is chromium. In some embodiments the co-metal is vanadium. In some embodiments the co-metal is titanium. In some embodiments the co-metal is scandium. In some embodiments the co-metal is the lanthanoid other than lanthanum. In some embodiments the palladium hydrogenation catalyst contains two co-metals. In some embodiments the two co-metals are lanthanum and any one of yttrium, nickel, zinc, copper, manganese, cobalt, iron, chromium, vanadium, titanium, scandium, and the lanthanoid other than lanthanum. In some embodiments the two co-metals are nickel and any one of yttrium, zinc, copper, manganese, cobalt, iron, chromium, vanadium, titanium, scandium, and the lanthanoid other than lanthanum. In some embodiments the two co-metals are yttrium and any one of zinc, copper, manganese, cobalt, iron, chromium, vanadium, titanium, scandium, and the lanthanoid other than lanthanum. In some embodiments the two co-metals are zinc and any one of copper, manganese, cobalt, iron, chromium, vanadium, titanium, scandium, and the lanthanoid other than lanthanum. In some embodiments the two co-metals are copper and any one of manganese, cobalt, iron, chromium, vanadium, titanium, scandium, and the lanthanoid other than lanthanum. In some embodiments the two co-metals are manganese and any one of cobalt, iron, chromium, vanadium, titanium, scandium, and the lanthanoid other than lanthanum. In some embodiments the two co-metals are cobalt and any one of iron, chromium, vanadium, titanium, scandium, and the lanthanoid other than lanthanum. In some embodiments the two co-metals are iron and any one of chromium, vanadium, titanium, scandium, and the lanthanoid other than lanthanum. In some embodiments the two co-metals are chromium and any one of vanadium, titanium, scandium, and the lanthanoid other than lanthanum. In some embodiments the two co-metals are vanadium and any one of titanium, scandium, and the lanthanoid other than lanthanum. In some embodiments the two co-metals are titanium and any one of scandium and the lanthanoid other than lanthanum. In some embodiments the two co-metals are scandium and the lanthanoid other than lanthanum. In some embodiments the palladium hydrogenation catalyst contains three or more co-metals. In some embodiments the co-metals are as described later in any one of the Examples.

The acidic mesoporous carbon support useful in the present invention can be characterized by its intrinsic acidity. Preferably a 1 wt % suspension of the acidic mesoporous carbon support has a potential of hydrogen (pH) of less than or equal to pH 6 (e.g., from pH 2 to pH 6), in some embodiments from pH 3 to pH 5, and more preferably from pH 3.5 to pH 4.5 (e.g., about pH 4).

Preferably the acidic mesoporous carbon support is characterizable as having a percent mesoporosity of greater than 25%, more preferably greater than 30%, and still more preferably greater than 40%. Larger values of percent mesoporosity are generally better but in some embodiments it can be desirably to balance it by maintaining a high BET surface area. In some embodiments the mesopore surface area used to calculate the percent mesoporosity is a mesopore surface area of 400 m²/g or greater, more preferably 500 m²/g or greater, and still more preferably 600 m²/g or greater. While the higher the mesopore surface area the better for this invention, in some embodiments the mesopore surface area is 1000 m²/g or less. In some embodiments the mesopore surface area is 700 m²/g or less. In some embodiments the mesopore surface area is as measured and described later in any one of the Examples.

In some embodiments the BET surface area used to calculate the percent mesoporosity is a BET surface area of 1000 m²/g or greater, more preferably 1200 m²/g or greater, and still more preferably 1300 m²/g or greater. While the higher the BET surface area the better for this invention, in some embodiments the BET surface area is 2000 m²/g or less. In some embodiments the BET surface area is 1500 m²/g or less. In some embodiments the BET surface area is as measured and described later in any one of the Examples.

Preferred structural forms of the aforementioned acidic mesoporous carbon support are activated carbons, graphite, carbon blacks, and multi-walled carbon nanotubes. In some embodiments the structural form is activated carbon. In some embodiments the structural form is carbon black. In some embodiments the structural form is graphite. In some embodiments the structural form is a fullerene, preferably a multi-walled carbon nanotube.

The invention advantageously provides high yields of and greater molar selectivities for the polyol ether as described later herein. The invention process employs a 3-fold or greater excess of the polyol relative to the amount of the carbonyl compound. In some embodiments the amount of polyol is characterizable by a molar ratio of the polyol to the carbonyl compound that is greater than or equal to 4 moles of polyol per 1 mole of the carbonyl compound (≧4:1). In some embodiments the invention process employs an excess amount of the polyol relative to the amount of the carbonyl compound wherein the excess amount is characterizable by a molar ratio of the polyol to the carbonyl compound that is greater than 5 moles of polyol per 1 mole of carbonyl compound (>5:1). Such a >5:1 molar ratio increases selectivity for a mono-ether form of the polyol ether over a di-ether form of the polyol ether. Without being bound by theory, it is believed that use of the excess amount of the polyol leads to higher yields of the polyol ether (particularly the mono-ether form thereof) than if the amount of polyol relative to the amount of the carbonyl compound is 5:1 or less (e.g., 4:1, 3:1, 2:1, or less preferably, 1:1). The higher yields of the mono-ether form of the polyol ether due to the excess amount (i.e., >5:1) are believed to be due, at least in part, to creation of reaction conditions that reduce or eliminate by-product formation. In some embodiments the molar ratio of the polyol to the carbonyl compound is at least 6:1, in other embodiments at least 7:1, in still other embodiments at least 8:1, and in yet other embodiments at least 9:1. In some embodiments the molar ratio is about 10:1. There is no particular upper limit on the amount of excess polyol that is used, especially since the polyol can be recycled and reused. While it is believed the higher the molar ratio the higher the yields, in some embodiments practical considerations can place an upper limit on the molar ratio. In some embodiments the polyol to carbonyl compound molar ratio does not exceed 100:1, more preferably does not exceed 50:1, and still more preferably does not exceed 30:1. More preferably the molar ratio is from 7:1 to 30:1, and even more preferred is a ratio of from 10:1 to 20:1. A molar ratio of 5:1 or less increases selectivity for the di-ether form of the polyol ether over the mono-ether form of the polyol ether. In some embodiments the molar ratio is as described later in any one of the Examples.

A portion of the excess polyol can thus function as a solvent. Preferably the invention process does not employ a solvent other than the polyol, which is employed in greater than 5 molar ratio excess relative to the carbonyl compound as described previously. In some embodiments, however, the invention process further employs a solvent such as, for example, diethyl ether, tetrahydrofuran, 1,4-dioxane, or an ethylene end-capped polyalkylene glycol. Preferably the solvent is substantially miscible with the polyol.

As previously mentioned, the invention advantageously provides high yields of the polyol ether. If the invention process is run for a sufficient time, preferably the process produces the polyol ether in greater than 70 percent yield, more preferably greater than 80 percent yield, still more preferably greater than 90 percent yield, and even more preferably greater than 92 percent yield, all based on the amount of the carbonyl compound. In some embodiments the percent yield is as described later in any one of the Examples.

As previously mentioned, the invention advantageously provides greater molar selectivities for the polyol ether. In some embodiments the molar selectivity is for the polyol ether over an alcohol by-product derived by reducing (adding hydrogen to) the carbonyl group of the carbonyl compound to give the corresponding alcohol by-product. In some embodiments the molar selectivity is for an intermediate acetal or ketal over the alcohol by-product. The intermediate acetal(s) or ketal(s) is derived from an in situ reaction of the carbonyl compound (R¹ is H and R² is not H in the case of the acetal(s) and both R¹ and R² are not H in the case of the ketal(s)) and the polyol with loss of a molecule of water, as illustrated later by a reaction scheme in Representative Procedure 1. Preferably the process is characterizable by a molar selectivity ratio of greater than 20:1, more preferably greater than 30:1, still more preferably greater than 50:1, and even more preferably greater than 70:1 for producing the polyol ether over a potential alcohol by-product of formula (IV). In some embodiments the molar selectivity ratio is as described later in any one of the Examples.

As mentioned previously, the selective hydrogenating conditions mean reaction conditions such as environmental parameters and other reaction features under which a hydrogenation reaction is conducted that preferentially yields the polyol ether, or preferentially yields the aforementioned acetal(s) or ketal(s) intermediates, or preferably both. Examples of the environmental parameters are pressure, temperature, catalyst loading (as described previously), and presence or absence of ancillary ingredients such as, for example, solvent. Preferably pressure is from ambient pressure (i.e., 14 pounds per square inch (psi), i.e., 100 kilopascals (kPa)) to 2000 psi (14,000 kPa). In some embodiments the pressure is from 50 psi (350 kPa) to 1000 psi (7000 kPa). In some embodiments the pressure is from 100 psi (690 kPa) to 500 psi (3500 kPa). In some embodiments, e.g., wherein the carbonyl compound is a sterically unhindered aldehyde, the pressure is 250 psi (1700 kPa) or less. In some embodiments the pressure is as described later in any one of the Examples.

Preferably the temperature is from ambient temperature (i.e., 24 degrees Celsius (° C.)) to 300° C. More preferably the temperature is 250° C. or less and still more preferably 220° C. or less. Also more preferably the temperature is 100° C. or more, and still more preferably 150° C. or more. In some embodiments the temperature is as described later in any one of the Examples.

Examples of the other reaction features of the selective hydrogenating conditions are concentrations of reaction ingredients, presence or absence of additives, and reaction time. Concentrations of the carbonyl compound and palladium hydrogenation catalyst in the polyol depend upon how much excess polyol is employed. The invention process will work with any concentrations of the carbonyl compound and palladium hydrogenation catalyst and such concentrations are not critical to the invention process. In some embodiments the concentrations are as described later in any one of the Examples.

In some embodiments the invention process is conducted in the absence of additives. That is, the invention process contacts ingredients consisting essentially of the polyol, carbonyl compound, hydrogen gas, and palladium hydrogenation catalyst. Use of additives is not critical to the invention process. In some embodiments the invention process further employs at least one additive, which preferably is an acidic co-catalyst comprising a Brønsted acid or Lewis acid, more preferably a weakly acidic Brønsted acid (i.e., a protic acid having a pKa greater than or equal to 3 (i.e., ≧3) and less than or equal to pKa 6 (i.e., ≦pKa 6) or weakly acidic Lewis acid. In some embodiments the additives are as described later in any one of the Examples.

Preferably the invention process produces the at least 30% yield of the polyol ether within 12 hours, more preferably within 6 hours, still more preferably within 4 hours, and even more preferably within 2 hours reaction time. Preferably the invention process produces at least 50% yield, more preferably at least 70%, still more preferably at least 80%, and even more preferably at least 90% yield of the polyol ether within 24 hours reaction time. More preferably the invention process produces the aforementioned yields within 6 hours, and still more preferably 4 hours reaction time. In some embodiments the yields and reaction times are as described later in any one of the Examples.

In some embodiments the invention process further comprises purifying the polyol ether. The polyol ethers can be purified by conventional means such as, for example, liquid/liquid extraction, fractional distillation, gas chromatography, high performance liquid chromatography, or a combination thereof (e.g., liquid/liquid extraction followed by fractional distillation of an extract therefrom). Preferably the purification separates the polyol ether(s) from at least one of the polyol, carbonyl compound, and any alcohol by-product from a reduction of the carbonyl compound. More preferably the purification separates the polyol ether(s) from at least a substantial amount (>90%) of the polyol, and still more preferably from at least the substantial amount of the polyol and separates polyol ether(s) that are polyol monoethers(s) from polyol ether(s) that are polyol diether(s). In some embodiments the purification is as described later in any on of the Examples.

For example for glycerol ethers, preferred methods of purifying glycerol ethers include:

For C3-C4 glycerol ethers (e.g., glycerol 1-propyl or 2-butyl ethers) and lower glycerol ethers (i.e., glycerol ethyl or methyl ethers) purification preferably comprises direct fractional vacuum distillation; since such monoethers and diethers typically have lower by than by of glycerol, monoethers and diethers are separable by fractional distillation;

For C5 glycerol ethers, monoethers and diethers typically have by lower than by of glycerol, but their separation by direct distillation can be challenging; so preferably diethers are first removed by extraction with heptane (or similar solvent), and then monoethers are distilled off from the remainder after extraction;

For C6 and higher (e.g., C7) glycerol ethers, the reaction mixture typically forms two phases after the reaction is completed; so preferably the monoethers/diethers are extracted (or decanted) first, concentrated, and then fractionally distilled, the extracted (or decanted) and concentrated monoethers and diethers being separable by vacuum distillation; such monoethers and diethers typically by have similar to or higher than by of glycerol.

### Materials and General Methods

Acidic mesoporous carbon support: BG-HHM carbon powder (amorphous) having a mesopore surface area (t-plot) of about 520 m²/g obtained from Calgon Carbon Corporation, Pittsburgh, Pa., USA.

### Surface Area Measurement Protocols for Brunauer-Emmett-Teller (BET) Surface Area and Mesopore Surface Area:

Sample preparation: Dry and degas samples before analysis as follows. After loading in an analysis tube and securing to the analysis tube a self-sealing cap (e.g., a ASAP 2020 TranSeal™ transfer seal, Micromeritics Instrument Corporation, Norcross, Ga., USA), connect a sample to a vacuum line and heat the sample for at least 4 hours (and up to 24 hours) while monitoring pressure. The heating temperature utilized should be high enough to fully dehydrate the sample without causing degradation or structural changes of the sample. For a polymeric material sample, use a heating temperature below the polymer's melting point (Tₘ) and preferably below its glass transition temperature (T_{g}). Silicas are frequently dried at 150° C. to 250° C.; aluminas at >250° C.; and zeolites at temperatures to 350° C. to insure removal of the last traces of moisture. Back fill the resulting heat activated sample with helium gas to atmospheric pressure. Transfer the resulting back filled sample to the analysis station of a Micromeritics ASAP 2405 series instrument without allowing atmospheric moisture to contact the back filled sample.

Measure the Brunauer-Emmett-Teller (BET) surface area in a manner similar to ASTM D-3663 and D-4222, from the physisorption of nitrogen at liquid nitrogen temperature 77 degrees Kelvin using the Micromeritics ASAP 2405 series instrument where both adsorption and desorption branches of isotherms are obtained. Initially equilibrate the back filled sample under vacuum, ensuring the back-filled sample is leak tight. Under the instrument's computer control dose nitrogen gas onto the sample in a stepwise fashion to predetermined relative pressures. Monitor pressure until an equilibrium value is maintained and recorded. The period of time which the instrument monitors the pressure between subsequent readings to ensure equilibrium is achieved is referred to as the equilibration interval. After equilibrium is obtained, admit another dose of nitrogen gas to the sample in order to move to a next relative pressure in the isotherm. After recording measured data for a full adsorption/desorption isotherm, mathematically generate all subsequent analyses from the measured data as follows.

Initially examine BET surface area data, micropore/external (mesopore) surface area data, and total and micropore volume data from the instrument's Summary Report to ascertain if the values are reasonable based upon knowledge of the sample. Examine the adsorption/desorption isotherm for:

General appearance including shape (isotherm type), volume adsorbed at lowest (about P/P₀=0.05) and highest (about P/P₀=0.99) P/P₀ points;

Closure of desorption and adsorption data at P/P₀<0.4. (Lack of closure indicates that there could be a slow leak from the atmospheric side of the instrument's manifold while a crossing of the desorption branch below the adsorption branch indicates a leak into the vacuum side of the manifold;

General appearance of adsorption/desorption hysteresis (if present); shape of the hysteresis provides information regarding geometry of the pore system;

Examine the tabular data from the instrument's isotherm analysis log and look to verify that the measured saturation pressures for nitrogen gas do not vary more than about 5% during course of the analysis. (This ensures that the bath temperature did not increase during the measurement run, which increase could be a problem for long runs (>36 hours).

### Next Examine Bet Statistics and Graph:

BET surface area value should be >5 m²/g and ensure that at least 3 m² is utilized for the analysis, i.e., (surface area)×(sample weight)>3 m²;

Correlation coefficient should be >0.999;

C value should be positive for test to be meaningful. (Certain microporous materials (not instant mesoporous carbon support) such as zeolites give negative C values indicating that the BET analysis is not meaningful for these materials although it is still often reported.);

Graph: P/P₀ of 0.05-0.20 is the range over which the equation is valid. (There should be no discernible adsorption features (transition) in this region);

t-Plot Statistics And Graph:

Micropore volume: positive value is meaningful especially when >0.02 cm³/g, negative value is equivalent to zero;

Correlation coefficient>0.999;

Micropore and external (mesopore) surface area: micropore area is calculated from micropore volume assuming spherical pores and external (mesopore) area calculated as the difference between the BET and micropore surface areas.

Graph: the thickness points utilized for analysis should be between about 3.5 Å to 7 Å. Lower values could lie in the micropore adsorption regime if present. A positive y-axis intercept indicates presence of microporosity. The plotted curve exhibits a so-called “roll-over” due to saturation at larger t values (higher pressures) for materials containing micropores. The points chosen for the analysis should be in the saturated region.

### BJH Desorption Pore Distribution Graph

Pore diameter at peak: analysis is only meaningful up to pore diameters of 500 Å to 1000 Å;

Pore volume at peak: maximum pore volume at peak should be greater than about 0.1 cm³/g to be meaningful.

Shape and width of distribution: note that analysis is invisible to pores having diameters greater than 500 Å to 1000 Å (pores having diameters greater than 1000 Å can be referred to as macropores).

### Examples of Acidic Mesoporous Carbon Supports Useful in the Instant Invention are Listed Below in Table A.

TABLE A
useful acidic mesoporous carbon supports in order of decreasing % mesoporosity:

| | | | % mesoporosity | |
|---|---|---|---|---|
| | BET | Mesopore surface | (mesopore | |
| | surface | area from t-plot | surface area/BET | |
| Carbon support | area (m²/g) | (external) (m²/g) | surface area) | pH |
| Hyperion MWCNT* carbon | 406 | 325 | 80 | 6 |
| Calgon BG-HHM carbon** | 1383 | 525 | 38 | 4 |
| acid washed Calgon BD | 525 | 50 | 9.5 | 4 |
| carbon (Calgon Carbon | | | | |
| Corp., Pittsburgh, | | | | |
| Pennsylvania, USA) | | | | |
| acid washed Calgon WPX | 773 | 75 | 9.7 | 4 |
| carbon | | | | |
| acid washed Calgon | 1437 | 50 | 3.5 | 4 |
| coconut carbon | | | | |

*MWCNT means multi-walled carbon nanotube;
**Calgon BG-HHM carbon is preferred over the acidic mesoporous carbon supports listed in Table A.
Preferred protocols for preparing the palladium hydrogenation catalyst

Preferably the palladium hydrogenation catalyst has been prepared by impregnation or deposition-adsorption of wetted PdCl₂ (e.g., H₂PdCl₄) or a wetted mixture of PdCl₂ and a co-metal chloride, respectively, on and into the acidic mesoporous carbon support to give a dry impregnated or deposited-adsorbed material, followed by an activating reduction of the impregnated or deposited-adsorbed material so as to produce the palladium hydrogenation catalyst, wherein the co-metal is as defined previously. Preferably the activating reduction is performed by flowing hydrogen gas over the impregnated or deposited-adsorbed material in a tube at an elevated temperature (e.g., 200° C.). Alternatively (less preferably) the activating reduction is performed by forming a slurry of the dry impregnated or deposited-adsorbed material in water and contacting the slurry with a hydride reagent such as sodium borohydride at ambient temperature (e.g., 24° C.).

Perform a preferred impregnation using an incipient wetness technique analogous to that illustrated later in Examples 1 and 2. Perform a preferred deposition-adsorption method analogous to that illustrated later in Example 3.

### pH Measurement Procedure: For Measuring pH of the Palladium Hydrogenation Catalyst or Acidic Mesoporous Carbon Support

Suspend 1 gram (g) of a palladium hydrogenation catalyst in 20 milliliters (mL) of deionized water at 20° C., stir the resulting 5 wt % suspension at room temperature for 5 minutes, and measure its pH with a calibrated pH meter.

### Catalyst Activation Procedure: for Activating Dried Palladium Hydrogenation Catalysts

Prior to use in a process, activate a dried palladium hydrogenation catalyst by placing 1.0 g sample thereof in a quartz U-tube reactor within a furnace. Flow 5 volume percent (vol %) hydrogen gas in nitrogen gas over the sample at a flow rate of 30 milliliters per minute (mL/min). Raise temperature of the furnace to 200° C. at a rate of 2° C. per minute, and then hold temperature at 200° C. for 2 hours. Cool the resulting solid back down to 25° C. under the 5 volume % hydrogen to give an activated form of the palladium hydrogenation catalyst.

### Carbon Monoxide (CO) Chemisorption Procedure: Characterization of Activated Palladium Hydrogenation Catalyst

Once the palladium hydrogenation catalyst has been activated, one can measure percent dispersion of the palladium hydrogenation catalyst. Percent dispersion indicates an amount of palladium metal atoms in the palladium hydrogenation catalyst that are available for catalyzing a hydrogenation. The measurement uses chemisorption of carbon monoxide (CO), which CO forms a 1:1 stoichiometric complex with the available palladium metal atoms. The percent dispersion is equal to (available palladium metal atoms divided by total number of palladium atoms) times 100. A Micromeritics 2010 unit is utilized to measure the volumetric uptake of CO by the palladium samples. The reduced samples are treated on the unit under a hydrogen flow to 150° C. to remove any adsorbed water and oxide layers that may have formed on the palladium. Two adsorption isotherms are sequentially performed with a 30 minute evacuation between them. The difference between the two isotherms represents the amount of CO chemisorbed on the sample. Utilizing an adsorption complex stoichiometry of 1:1 for CO to palladium and knowing the weight % loading of palladium allows calculation of the % dispersion of the palladium metal.

Representative Procedure 1: Synthesizing 2-(pentyloxy)-1,3-propanediol and/or Racemic 3-(pentyloxy)-1,2-propanediol (i.e., 1-pentoxy-2,3-propanediol) and/or 1,3-bis(pentyloxy)-2-propanol and Racemic 2,3-bis(pentyloxy)-1-propanol (polyol ethers) from Glycerol (polyol), Valeraldehyde (Carbonyl Compound) and Hydrogen Gas in the Presence of a Hydrogenation Catalyst

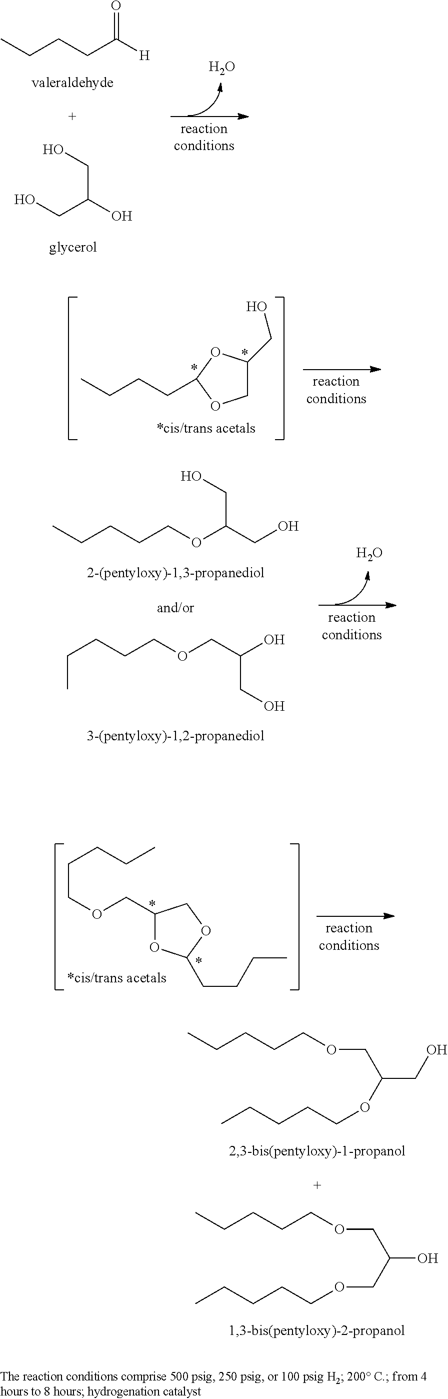

With stirring charge into a high pressure 150 mL volumed autoclave reactor glycerol (27.6 g, 0.3 mol) and 0.13 g of a hydrogenation catalyst (catalyst loading 5 wt % relative to valeraldehyde; other catalyst loadings of 2.5 wt %, 3.2 wt %, 10 wt %, or 15 wt % can be used as the case may be; see Table 2 later for details.) Seal and flush the reactor with hydrogen gas at 100 pounds per square inch or psi; 690 kilopascals (kPa) three times with stirring. Then add valeraldehyde (2.6 g, 0.03 mol by syringe, purge the reactor again two times with stirring as before, and then initially pressurize the reactor to 100 psi (690 kPa) or 500 psi (3400 kPa) with hydrogen gas (H₂ (g)) as the case may be; see Table 2 later for details. Then heat the reactor to 200° C. On attaining 200° C., adjust pressure of the hydrogen gas to either 1000 pounds per square inch gauge (psig, 6900 kPa), 500 psig (3400 kPa), 250 psig (1700 kPa), or 100 psig (690 kPa) as the case may be; see Table 2 later for details. Continue stirring the reaction mixture for 4 hours or 6 hours as the case may be; see Table 2 later for details. After 4 hours or 6 hours, as the case may be, cool the reactor and its contents, and release residual hydrogen gas. Analyze a sample of the cooled reaction mixture by gas chromatography as described previously to measure amounts of 2-(pentyloxy)-1,3-propanediol and/or racemic 3-(pentyloxy)-1,2-propanediol and/or 1,3-bis(pentyloxy)-2-propanol and racemic 2,3-bis(pentyloxy)-1-propanol and any intermediates and by-products (e.g., 1-pentanol and dipentyl ether).

Non-limiting examples of the present invention are described below that illustrate some specific embodiments and aforementioned advantages of the present invention. Preferred embodiments of the present invention incorporate one limitation, and more preferably any two, limitations of the Examples, which limitations thereby serve as a basis for amending claims.

### EXAMPLE(S) OF THE PRESENT INVENTION

### Example 1 (EX-1)

### Preparation of 5 wt % Palladium on Acidic Mesoporous Carbon Support by Incipient-Wetness Impregnation

Impregnate 1.00 g of the aforementioned Calgon BG-HHM carbon powder (that has been dried beforehand at 110° C. for 4 hours) to incipient wetness using 1.08 g of a solution containing 5 wt % Pd as PdCl₂ dissolved in 5 wt % aqueous HCl. Allow the resulting wet solid to partially dry in a fume hood at room temperature overnight, and then place the partially dried solid in a 110° C. oven for 4 hours to give a dried solid that is the catalyst of EX-1.

Activate the catalyst of EX-1 according to the aforementioned Catalyst Activation Procedure to give an activated form of the catalyst of EX-1. According to the aforementioned CO Chemisorption procedure, the activated form of the catalyst of EX-1 adsorbs 4.98 cubic centimeters (cm³) standard temperature (25° C.) and pressure (101 kPa) CO per gram thereof, which adsorption translates to a 47.3% Pd metal dispersion, based on total amount of Pd employed, that is available for reaction on the acidic mesoporous carbon support.

### Example 2 (EX-2)

### Preparation of 1 wt % Palladium (Pd)-5 wt % Lanthanum (La) on Acidic Mesoporous Carbon Support by Incipient-Wetness Impregnation

Impregnate 3.05 g of the Calgon BG-HHM carbon powder (that has been dried beforehand at 110° C. for 4 hours) to incipient wetness using 3.0 g of an aqueous solution containing 5 wt % La (as LaCl₃.7H₂O). Allow the resulting wet solid to partially dry in a fume hood at room temperature overnight, and then place the partially dried solid in a 110° C. oven for 4 hours to give a dried solid. Impregnate a 2.4 g portion of this dried solid with 2.4 g of a solution containing 1 wt % PdCl₂ dissolved in 1 wt % aqueous HCl. Allow the resulting wet solid to partially dry in a fume hood at room temperature overnight, and then place the partially dried solid in an 110° C. oven for 4 hours to give a dried solid that is the catalyst of EX-2.

Activate the catalyst of EX-2 according to the aforementioned Catalyst Activation Procedure to give an activated form of the catalyst of EX-2. According to the aforementioned CO Chemisorption procedure, the activated form of the catalyst of EX-2 adsorbs 0.63 cm³ standard temperature and pressure CO per gram thereof, which adsorption translates to a 29.8% Pd metal dispersion that is available for reaction on the acidic mesoporous carbon support. The percent Pd metal dispersion is based on total amount of Pd employed.

### Example 3 (EX-3)

### Preparation of 5 wt % Palladium on Acidic Mesoporous Carbon Support by Solution Deposition-Adsorption

With stirring slurry 5.1 g of the Calgon BG-HHM carbon powder in 100 mL of distilled water. To the resulting slurry add 5.1 g of a solution containing 5 wt % PdCl₂ dissolved in 5 wt % aqueous HCl, and continue stirring for 18 hours. Filter the resulting solid, and wash the resulting filtercake with 200 mL of distilled water. Allow the resulting wet solid to partially dry in a fume hood at room temperature overnight and, then place the partially dried solid in an 110° C. oven for 4 hours to give a dried solid that is the catalyst of EX-3. Activate the catalyst of EX-3 according to the aforementioned Catalyst Activation Procedure to give an activated form of the catalyst of EX-3.

Examples 4 to 16: prepare catalysts in a manner similar to the procedure of any one of Examples 1 to 3, as the case may be, except use NiCl₂ instead of LaCl₃ in EX-10 and use relative amounts of metal halide(s) to the Calgon BG-HHM carbon powder and relative amounts of PdCl₂ to either NiCl₂ or LaCl₃, as the case may be in EX-10 to EX-14, to arrive at the catalyst compositions; see Table 1 below for details.

TABLE 1
catalysts of Examples 4 to 15 and their preparation

| | | | Preparation |
|---|---|---|---|
| Example | | | Method of |
| Number | Catalyst Composition | Preparation Technique | (Example No.) |
| 4 | 5 wt % Pd/C* | Incipient wetness | 1 |
| 5 | 5 wt % Pd/C* | Incipient wetness | 1 |
| 6 | 2.5 wt % Pd/C* | Incipient wetness | 1 |
| 7 | 5 wt % Pd/C* | Incipient wetness | 1 |
| 8 | 2.5 wt % Pd/C* | Incipient wetness | 1 |
| 9 | 5 wt % Pd/C* | Incipient wetness | 1 |
| 10 | 1 wt % Pd-2.5 wt % Ni/C* | 2-step Incipient wetness, | 2 |
| | | first Ni then Pd | |
| 11 | 5 wt % Pd-5 wt % La/C* | 2-step Incipient wetness, | 2 |
| | | first La then Pd | |
| 12 | 5 wt % Pd-5 wt % La/C* | 2-step Incipient wetness, | 2 |
| | | first La then Pd | |
| 13 | 1 wt % Pd-5 wt % La/C* | 2-step Incipient wetness, | 2 |
| | | first La then Pd | |
| 14 | 1 wt % Pd-5 wt % La/C* | 2-step Incipient wetness, | 2 |
| | | first La then Pd | |
| 15 | 5 wt % Pd/C* | Solution-adsorption | 3 |
| 16 | 2.5 wt % PC-5 wt % La/C* | 2-step Incipient wetness, | 2 |
| | | first La then Pd | |

wherein C* means acidic mesoporous carbon support.

According to the aforementioned pH Measurement Procedure, suspensions in water of catalysts of each of Examples 1 to 15 independently have a pH of about pH 4.

### Examples A to R (EX-A to EX-R)

### Synthesizing 2-(pentyloxy)-1,3-propanediol and/or Racemic 3-(pentyloxy)-1,2-propanediol and/or 1,3-bis(pentyloxy)-2-propanol and Racemic 2,3-bis(pentyloxy)-1-propanol from Glycerol, Valeraldehyde and Hydrogen Gas in the Presence of a Hydrogenation Catalyst

Repeat the aforementioned Representative Procedure 1 eighteen times, each time using a different one of the catalysts of EX-1 and EX-3 to EX-15 (employ different portions of catalyst of EX-3 four times and different portions of catalyst of EX-7 two times) as shown below in Table 2. In Table 2, reaction time is 4 hours except for EX-C.

TABLE 2
gas chromatography results of synthesis of 2-(pentyloxy)-1,3-propanediol and/or racemic 3-
(pentyloxy)-1,2-propanediol (polyol monoethers) and/or 1,3-bis(pentyloxy)-2-propanol and
racemic 2,3-bis(pentyloxy)-1-propanol (polyol diethers) according to EX-A to EX-R.

| | | | | | | | Selectivity |
|---|---|---|---|---|---|---|---|
| | | | | | | | for polyol |
| | | | | | GC | GC | mono- |
| | | | | GC Yield | Combined | Combined | ethers/ |
| React. | EX No. | React. | Cat. | 1- | Yield | Yield of | polyol |
| EX | (Cat. | Press. | Load. | pentanol | cis/trans | polyol | diether |
| No. | Comp.) | (psig) | (wt %) | (%) | acetals (%) | ethers (%) | (%/%) |
| EX-A | EX-3 | 500 | 5 | 3.4 | 0.5 | 91.2 | 9.6 |
| | (5 wt % | | | | | | |
| | Pd/C*) | | | | | | |
| EX-B | EX-3 | 250 | 5 | 4.0 | 0.4 | 83.3 | 6.4 |
| | (5 wt % | | | | | | |
| | Pd/C*) | | | | | | |
| EX-C | EX-3 | 100 | 5 | 2.4 | 11.9 | 74.4 | 12 |
| | (5 wt % | | | | | | |
| | Pd/C*) | | | | | | |
| EX-D | EX-1 | 1000 | 10 | 2.4 | 0.74 | 90.6 | 8.9 |
| | (5 wt % | | | | | | |
| | Pd/C*) | | | | | | |
| EX-E | EX-4 | 1000 | 10 | 3.4 | Not | 92.2 | 8.3 |
| | (5 wt % | | | | detected | | |
| | Pd/C*) | | | | | | |
| EX-F | EX-5 | 1000 | 10 | 6.6 | 0.3 | 85.7 | 13 |
| | (5 wt % | | | | | | |
| | Pd/C*) | | | | | | |
| EX-G | EX-6 | 1000 | 10 | 1.3 | 1.9 | 93.3 | 11 |
| | (2.5 wt % | | | | | | |
| | Pd/C*) | | | | | | |
| EX-H | EX-7 | 1000 | 10 | 3.6 | 0.4 | 91.7 | 9.1 |
| | (5 wt % | | | | | | |
| | Pd/C*) | | | | | | |
| EX-I | EX-7 | 1000 | 5 | 1.7 | 0.25 | 89.0 | 8.1 |
| | (5 wt % | | | | | | |
| | Pd/C*) | | | | | | |
| EX-J | EX-8 | 1000 | 10 | 2.8 | 1.7 | 93.3 | 8.5 |
| | (2.5 wt % | | | | | | |
| | Pd/C*) | | | | | | |
| EX-K | EX-9 | 1000 | 3.2 | 4.8 | 0.5 | 86.3 | 11 |
| | (5 wt % | | | | | | |
| | Pd/C*) | | | | | | |
| EX-L | EX-10 | 1000 | 15 | 7.1 | 1.8 | 84.1 | 12 |
| | (1 wt % Pd- | | | | | | |
| | 2.5 wt % | | | | | | |
| | Ni/C*) | | | | | | |
| EX-M | EX-11 | 1000 | 5 | 4.9 | 2.0 | 85.9 | 9.9 |
| | (5 wt % Pd- | | | | | | |
| | 5 wt % | | | | | | |
| | La/C*) | | | | | | |
| EX-N | EX-12 | 1000 | 5 | 4.9 | 3.9 | 85.7 | 10.0 |
| | (5 wt % Pd- | | | | | | |
| | 5 wt % | | | | | | |
| | La/C*) | | | | | | |
| EX-O | EX-13 | 1000 | 15 | 4.3 | 9.7 | 83.7 | 19 |
| | (1 wt % Pd- | | | | | | |
| | 5 wt % | | | | | | |
| | La/C*) | | | | | | |
| EX-P | EX-14 | 1000 | 15 | 4.6 | 3.6 | 88.2 | 12 |
| | (1 wt % Pd- | | | | | | |
| | 5 wt % | | | | | | |
| | La/C*) | | | | | | |
| EX-Q | EX-3 | 1000 | 5 | 3.2 | 0.65 | 90.7 | 10.3 |
| | (5 wt % | | | | | | |
| | Pd/C*) | | | | | | |
| EX-R | EX-15 | 1000 | 5 | 3.6 | 1.4 | 92.2 | 9.0 |
| | (5 wt % | | | | | | |
| | Pd/C*) | | | | | | |

wherein “React. EX No.” means reaction Example Number; “EX No. (Cat. Comp.)” means catalyst Example Number and catalyst composition; “C*” means acidic mesoporous carbon support; “React. Press. (psig)” means reaction pressure in pounds per square inch gauge; “Cat. Load. (wt %)” means catalyst loading in weight percent relative to valeraldehyde; “GC Yield 1-pentanol (%)” means percent yield of 1-pentanol as determined by gas chromatography; “GC Combined Yield cis/trans acetals” means percent yield of the cis/trans acetals shown in both pairs of brackets in the scheme in Representative Procedure 1; “GC Combined Yield of polyol ethers (%)” means percent yield of 2-(pentyloxy)-1,3-propanediol; racemic 3-(pentyloxy)-1,2-propanediol; and 1,3-bis(pentyloxy)-2-propanol and racemic 2,3-bis(pentyloxy)-1-propanol; and “Selectivity for polyol mono-ethers/polyol diether (%/%)” means percent yield of 2-(pentyloxy)-1,3-propanediol and racemic 3-(pentyloxy)-1,2-propanediol (polyol monoethers) divided by percent yield of 1,3-bis(pentyloxy)-2-propanol and racemic 2,3-bis(pentyloxy)-1-propanol (polyol diethers).

### Example S

### Reuse a Single Batch of Invention Catalyst 2.5% Pd-5% La/C, (0.26 g, 10 wt % Relative to Valeraldehyde) in Five Consecutive Reaction Runs

Perform the following reaction procedure 5 times except successively reuse a single batch of invention catalyst from one run of the reaction procedure to the next run of the procedure.

Run 1: in a first run glycerol (27.6 g, 0.3 mol) and an invention catalyst 2.5% Pd-5% La/C (Example 16; 0.26 g, 10 wt % relative to valeraldehyde) are charged to a 150 mL Parr reactor containing a bottom opening with a filter. The bottom valve is closed. The reactor is sealed and purged with hydrogen at 100 psi three times with stirring. Then valeraldehyde (2.60 g, 0.03 mol) is added by syringe and the reactor is additionally purged with hydrogen at 100 psi two times with stirring. Then 100 psi of hydrogen is charged, the reactor is quickly heated to 200° C. and hydrogen pressure is set at 250 psi. The samples are taken every 2 hours by syringe, after cooling the reactor with cold water via an internal coil and releasing the hydrogen pressure, and analyzed by GC. After 6 hours, the reaction mixture is cooled and discharged via the bottom filter, retaining the catalyst inside the reactor.

Run 2: in a second run, glycerol (27.6 g, 0.3 mol) and valeraldehyde (2.60 g, 0.03 mol) are charged to the reactor by syringe, the reactor is sealed and purged with hydrogen at 100 psi three times with stirring. Then 100 psi of hydrogen is charged, the reactor is quickly heated to 200° C. and hydrogen pressure is set at 250 psi. The samples are taken every 2 hours by syringe after cooling the reactor with cold water via an internal coil and releasing the hydrogen pressure and analyzed by GC. The mixture was discharged after 8 hours.

Runs 3 to 5: the procedure of Run 2 is repeated for three more consecutive runs, Runs 3 to 5, reusing catalyst obtained from Run 2, Run 3, and Run 4, respectively).

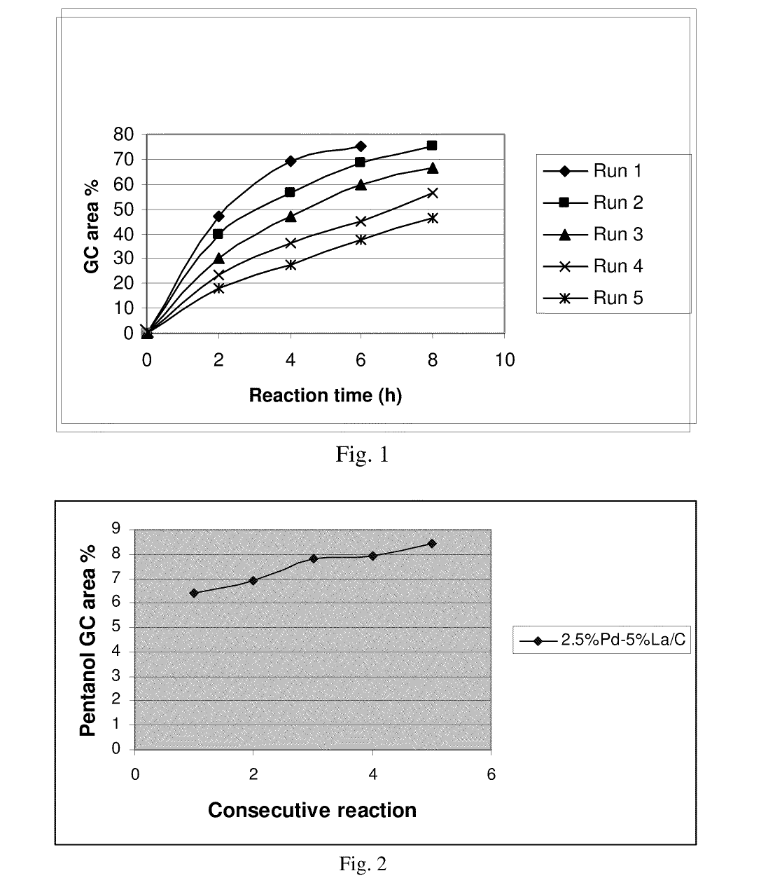

The total number of runs with the same batch of catalyst is five. The GC area % of glycerol monoethers (3-pentyloxy-1,2-propanediol+2-pentyloxy-1,3-propanediol) versus time for the five runs of Example S are shown in FIG. 1.

1-Pentanol is an undesirable aldehyde reduction byproduct in catalytic reductive etherification. Pentanol selectivity increases marginally for 2.5% Pd-5% La/C in Example S with every subsequent catalyst reuse as shown by results of pentanol GC area percent that are plotted in FIG. 2. Pentanol would increase substantially faster from one run to a next run with a non-invention, conventional 10 wt % Pd/C catalyst (data not shown).

### Example T

### Preparation and Purification of the Glycerol Propyl Ethers, 3-propoxy-1,2-propanediol and 2-propoxy-1,3-propanediol

Prepare a reaction mixture resulting from reductive etherification of propionaldehyde (14.52 g; 0.25 mol) with glycerol (230.2 g; 2.5 mol) using 0.726 g of invention catalyst 5 wt % Pd/C (prepared in a manner similar to that of Example 1; 5 wt % relative to the aldehyde) according to the manner of Representative Procedure 1. The reaction mixture contains 86% of 3-propoxy-1,2-propanediol and 2-propoxy-1,3-propanediol, 7.5% of 1,3-dipropoxy-2-propanol and 1,2-dipropoxy-3-propanol, and 0.7% of 1-propanol based on GC area %. Filter off the catalyst to give a filtrate, and wash the filtered catalyst with methanol (2×50 mL). Evaporate the methanol in vacuum, combine the resulting residue with the filtrate, and fractionally distill the resulting mixture in vacuum to yield 29.7 g (88%) of a purified mixture of 3-propoxy-1,2-propanediol and 2-propoxy-1,3-propanediol, boiling point (bp) 60° C. to 61° C. at a pressure of 0.15 millimeter of mercury (mm Hg; 20 pascals).

### Example U

### Preparation and Purification of the Glycerol Butyl Ethers, 3-butoxy-1,2-propanediol and 2-butoxy-1,3-propanediol

Prepare and purify a reaction mixture following the procedure of Example T except using butyraldehyde (18.03 g; 0.25 mol) with glycerol (230.2 g; 2.5 mol) using 0.90 g of 5 wt % Pd/C (prepared in a manner similar to that of Example 1; 5 wt % relative to the aldehyde). The reaction mixture contains 83.6% of 3-butoxy-1,2-propanediol and 2-butoxy-1,3-propanediol, 5.5% of 1,3-dibutoxy-2-propanol and 1,2-dibutoxy-3-propanol, and 1.2% of 1-butanol based on GC area %. Purification of the reaction mixture yields 31.1 g (84%) of a purified mixture of 3-butoxy-1,2-propanediol and 2-butoxy-1,3-propanediol, by 68° C. to 69° C./0.07 mm Hg (9 pascals).

### Example V

### Preparation and Purification of the Glycerol 3-methylbutyl ethers, 3-(3-methylbutoxy)-1,2-propanediol and 2-(3-methylbutoxy)-1,3-propanediol

Following Representative Procedure 1, prepare a reaction mixture resulting from reductive etherification of 3-methylbutanal (23.25 g; 0.27 mol) with glycerol (248.7 g; 2.7 mol) using 1.16 g of 5 wt % Pd/C (prepared in a manner similar to that of Example 1; 5 wt % relative to the aldehyde). The reaction mixture contains 82.9% of 3-(3-methylbutoxy)-1,2-propanediol and 2-(3-methylbutoxy)-1,3-propanediol, 6.2% of 1,3-bis(3-methylbutoxy)-2-propanol and 1,2-bis(3-methylbutoxy)-3-propanol, and 3% of 3-methylbutanol based on GC area %. Filter off the catalyst to give a filtrate, and wash the filtered catalyst with methanol (2×50 mL). Evaporate the methanol in vacuum, combine the resulting residue with the filtrate. Wash the combination with heptane (3×100 mL) to substantially remove glycerol di(3-methylbutyl)ethers into the heptane washes. Fractionally distill the washed combination in vacuum to yield 34.6 g (83%) of 3-(3-methylbutoxy)-1,2-propanediol and 2-(3-methylbutoxy)-1,3-propanediol, by 72° C. to 74° C./0.08 mm Hg (11 pascals). The glycerol di(3-methylbutyl)ethers can be recovered by evaporating heptane from the washes.

### Example W

### Preparation and Purification of the Glycerol Heptyl Ethers, 3-(heptyloxy)-1,2-propanediol and 2-(heptyloxy)-1,3-propanediol

Following Representative Procedure 1, prepare a reaction mixture resulting from reductive etherification of heptanal (22.8 g; 0.2 mol) with glycerol (184.2 g; 2.0 mol) using 1.14 g of 5 wt % Pd/C (prepared in a manner similar to that of Example 1; 5 wt % relative to the aldehyde). The reaction mixture contains 76.2% of 3-(heptyloxy)-1,2-propanediol and 2-(heptyloxy)-1,3-propanediol and 9.6% of 1,3-bis(3-methylbutoxy)-2-propanol and 1,2-bis(3-methylbutoxy)-3-propanol based on GC area %. Filter off the catalyst to give a filtrate, and wash the filtered catalyst with methanol (2×50 mL). Evaporate the methanol in vacuum, and combine the resulting residue with the filtrate. Add 180 mL of water and extract the combination five times with diethyl ether (5×50 mL). Dry the combined diethyl ether phases (extracts) with sodium sulfate, evaporate the diethyl ether, and fractionally distill the resulting residue in vacuum to yield 28.7 g (75%) of 3-(heptyloxy)-1,2-propanediol and 2-(heptyloxy)-1,3-propanediol, by 91° C. to 93° C./0.06 mm Hg (8 pascals).

### Example X

### Preparation and Purification of 3-pentyloxy-1,2-propanediol and 2-pentyloxy-1,3-propanediol

Load valeraldehyde (86.13 g, 1 mol), glycerol (920.9 g, 10 mol), and 5% Pd/C catalyst (5 wt % relative to the aldehyde, 4.30 g) under nitrogen into a 2 L Parr reactor. Seal the reactor, and purge it with hydrogen gas three times at about 100 psi with stirring. Then charge the reactor with hydrogen gas (100 psi), quickly heat contents of the reactor to 200° C. with stirring, and set hydrogen pressure at 300 psi. After 10 hours at 200° C. and 300 psi of hydrogen, cool the reactor and vent remaining hydrogen gas to give a reaction mixture containing 84.6% of 3-pentyloxy-1,2-propanediol and 2-pentyloxy-1,3-propanediol, 9.1% of 1,3-bis(pentyloxy)-2-propanol and 1,2-bis(pentyloxy)-3-propanol, and 2.3% of 1-pentanol based on GC area %. Discharge the reaction mixture from reactor, and wash the reactor with methanol (1 L×2). Filter off the catalyst to give a filtered solution, and wash catalyst with the methanol reactor washings. Evaporate methanol in vacuum, and combine the residue with the filtered solution. Fractionally distill the resulting combined and filtered solution in vacuum at 70-73° C./0.05 mm Hg to give a crude product (135.8 g), which contains 3-pentyloxy-1,2-propanediol and 2-pentyloxy-1,3-propanediol along with 11.4% of 1,3-bis(pentyloxy)-2-propanol and 1,2-bis(pentyloxy)-3-propanol by GC area %. Dissolve a portion (101.5 g) of crude product in water (400 mL), and extract aqueous solution with heptane (100 mL×5). Evaporate water from the resulting heptane extracted aqueous solution in vacuum to give 93.0 g (77%) of a mixture of 3-pentyloxy-1,2-propanediol and 2-pentyloxy-1,3-propanediol of Example X, which mixture contains less than 1.5% of 1,3-bis(pentyloxy)-2-propanol and 1,2-bis(pentyloxy)-3-propanol byproducts. The dipentyl ethers of glycerol can be recovered by evaporating heptane from the washes.

### Example Y

### Preparation and Purification of 3-hexyloxy-1,2-propanediol and 2-hexyloxy-1,3-propanediol

Repeat reaction procedure of Example X except with hexanal (100.2 g, 1 mol), glycerol (920.9 g, 10 mol), and 5% Pd/C catalyst (5 wt % relative to the aldehyde, 5.01 g) to give a reaction mixture containing 79.5% of 3-hexyloxy-1,2-propanediol and 2-hexyloxy-1,3-propanediol, 8.6% of 1,3-bis(hexyloxy)-2-propanol and 1,2-bis(hexyloxy)-3-propanol, and 2.0% of 1-hexanol based on GC area %. Repeat reactor discharge, catalyst filtering, reactor washing, catalyst washing, methanol evaporating, and residue combining procedure to give a combined and filtered solution. Extract the combined and filtered solution with toluene (200 mL×10), combine toluene extracts, and evaporate toluene therefrom to give a residue. Fractionally distil the extract residue in vacuum, giving 123.6 g of 3-hexyloxy-1,2-propanediol and 2-hexyloxy-1,3-propanediol at 78° C./0.04 mm to 81° C./0.04 mm, which also contains 7.9% of 1,3-bis(hexyloxy)-2-propanol and 1,2-bis(hexyloxy)-3-propanol. Dissolve a portion of this product (115.0 g) in a mixture of acetonitrile (400 mL) and water (10 mL), and extract the resulting solution with heptane (100 mL×5). Evaporate acetonitrile and water from the resulting heptane extracted solution in vacuum to give 99.5 g (61%) of purified 3-hexyloxy-1,2-propanediol and 2-hexyloxy-1,3-propanediol of Example Y containing only about 1.1% of the 1,3-bis(hexyloxy)-2-propanol and 1,2-bis(hexyloxy)-3-propanol byproducts. The dihexyl ethers of glycerol can be recovered by evaporating heptane from the washes.

### Example Z

### Preparation and purification of 3-(2-ethylhexyloxy)-1,2-propanediol and 2-(2-ethylhexyloxy)-1,3-propanediol

Repeat the procedure of Example X except use 2-ethylhexanal (128.2 g, 1 mol), glycerol (920.9 g, 10 mol), and 5% Pd/C catalyst (5 wt % relative to the aldehyde, 6.41 g) and the following reaction conditions in the reactor: 200° C. with stirring, a hydrogen gas pressure of 500 psi, and 14 hours reaction time to give a reaction mixture containing 76.5% of 3-(2-ethylhexyloxy)-1,2-propanediol and 2-(2-ethylhexyloxy)-1,3-propanediol, 11.8% of 1,3-bis(2-ethylhexyloxy)-2-propanol and 1,2-bis(2-ethylhexyloxy)-3-propanol, and 2.0% of 2-ethylhexanol based on GC area %. Repeat the reactor discharge, catalyst filtering, reactor washing, catalyst washing, methanol evaporating, and residue combining procedure to give a combined and filtered solution having upper and lower phases. Separate the upper (product) phase, and extract the lower (glycerol) phase with toluene (300 mL×6). Evaporate the toluene extracts, and combine the resulting extract residue with the upper (product) phase to give a crude product (178.4 g). Fractionally distil a portion (128.4 g) of the crude product in vacuum to give 103.7 g (71%) of 3-(2-ethylhexyloxy)-1,2-propanediol and 2-(2-ethylhexyloxy)-1,3-propanediol, by 82-84° C./0.06 mm Hg.

As shown by the results in Table 2 and FIGS. 1 and 2, the invention process advantageously produces the polyol ether without a need for any added acid co-catalyst. The invention process reduces or avoids acid co-catalyst promoted condensation between two carbonyl compounds, polycondensation of polyols, deactivate certain hydrogenation catalysts, or a combination thereof. The invention process also advantageously produces the polyol ether in high yields (typically greater than 70% yield) and selectivities over by-products. The invention discovered that the acidic mesoporous carbon support having the aforementioned percent mesoporosity facilitates increased catalytic activity of the palladium hydrogenation catalyst compared to palladium catalysts on carbon that lack the acidity and percent mesoporosity features thereof, and even compared to palladium catalysts on mesoporous carbon that have been prepared by methods other than the instant impregnation or deposition-adsorption. The higher yields of and increased selectivities for the polyol ethers makes the invention process especially valuable in the preparation of polyol ethers for use as, for example, solvents, surfactants, wetting agents, emulsifying agents, lubricants, and intermediates for the preparation of surfactants.

While the invention has been described above according to its preferred embodiments, it can be modified within the spirit and scope of this disclosure. This application is therefore intended to cover any variations, uses, or adaptations of the instant invention using the general principles disclosed herein. Further, the instant application is intended to cover such departures from the present disclosure as come within the known or customary practice in the art to which this invention pertains and which fall within the limits of the following claims.

1. A process for making a polyol ether, the process comprising contacting together under selective hydrogenating conditions an excess amount of a polyol, an amount of a carbonyl compound, an excess amount of hydrogen gas, and a catalytic amount of a palladium hydrogenation catalyst on an acidic mesoporous carbon support so as to provide the polyol ether, wherein:
(a) the carbonyl compound is of formula (I):
R¹R²C═O (I)
wherein each of R¹ and R² independently is hydrogen atom (H), (C₁-C₅₀)alkyl, (C₂-C₅₀)alkenyl, (C₆-C₁₀)aryl-(C₁-C₅₀)alkyl, (C₆-C₁₀)aryl-(C₂-C₅₀)alkenyl-, or (C₃-C₁₂)cycloalkyl; or R¹ and R² together with the carbon atom to which they are both attached form a (C₃-C₁₂)cycloalkyl ring;
(b) the polyol is a compound of formula (II):
HO—[CH(R³)-Q-CR⁴(R⁵)—O]ₘ—H (II)
wherein m is an integer of from 1 to 2000;
each Q independently is a covalent bond, L, X, L-X, X-L, or L-X-L, wherein each L independently is (C₁-C₁₄)alkylene, (C₁-C₁₄)heteroalkylene, or (C₂-C₁₄)alkenylene; and each X independently is (C₃-C₁₂)cycloalkylene, (C₂-C₁₂)heterocycloalkylene, (C₆-C₁₀)arylene, or (C₁-C₁₀)heteroarylene;
each of R³, R⁴, and R⁵ independently is H, (C₁-C₂₀)alkyl, (C₆-C₁₀)aryl-(C₁-C₁₀)alkyl, or (C₃-C₁₂)cycloalkyl; or R⁴ and R⁵ are together with the carbon atom to which they are both attached form a (C₃-C₁₂)cycloalkyl ring;
(c) the polyol ether comprises a compound of formula (IIIa), (IIIb), or (IIIc):
R¹R²C(H)—O—[CH(R³)-Q-CR⁴(R⁵)—O]ₘ—H (IIIa),
HO—[CH(R³)-Q-CR⁴(R⁵)—O]ₘ—CHR¹R² (IIIb), or
R¹R²C(H)—O—[CH(R³)-Q-CR⁴(R⁵)—O]ₘ—CHR¹R² (IIIc), or
a mixture of any two or more compounds of the formulas (IIIa), (IIIb), and (IIIc),
wherein m, Q, and R¹ to R⁵ are as defined previously; and each alkyl, alkylene, alkenyl, alkenylene, aryl, arylene, cycloalkyl, cycloalkylene, (C₁-C₁₄)heteroalkylene, and (C₂-C₁₂)heterocycloalkylene group independently is unsubstituted or substituted with from 1 to 10 substituent groups R^{S}, wherein each R^{S} is bonded to a carbon atom and independently is a hydroxyl (—OH), ═O, halo, di(C₁-C₂₀)alkylamino, (C₁-C₆)alkyl, —CHO (i.e., —C(═O)—H), —CO(C₁-C₆)alkyl (i.e., —C(═O)—(C₁-C₆)alkyl), —CO₂H, —CO₂(C₁-C₆)alkyl, —CON((C₁-C₆)alkyl)₂, (C₁-C₆)alkoxy, (C₂-C₆)alkynyl, or —SH;
(d) the palladium hydrogenation catalyst comprises palladium(0) or a palladium(0)-(co-metal) comprising palladium(0) in the presence of at least one co-metal, wherein the co-metal is lanthanum, yttrium, nickel, zinc, copper, manganese, cobalt, iron, chromium, vanadium, titanium, scandium, or a lanthanoid other than lanthanum; the palladium(0) or palladium(0)-(co-metal) being supported on a surface of the acidic mesoporous carbon support; the palladium hydrogenation catalyst having been prepared by impregnation or deposition-adsorption of a PdCl₂ or independently a PdCl₂ and a corresponding co-metal chloride, respectively, on and into the acidic mesoporous carbon support so as to give an impregnated or deposited-adsorbed material, followed by an activating reduction of the impregnated or deposited-adsorbed material so as to produce the palladium hydrogenation catalyst;
(e) the acidic mesoporous carbon support is characterizable as having a percent mesoporosity of greater than 15%, wherein percent mesoporosity is equal to 100 times mesopore surface area of the acidic mesoporous carbon support (square meters per gram) divided by Brunauer-Emmett-Teller surface area of the acidic mesoporous carbon support (square meters per gram);
(f) the excess amount of the polyol is relative to the amount of the carbonyl compound and is characterizable by a molar ratio of the polyol to the carbonyl compound that is greater than or equal to 3 to 1 (≧3:1); and
(g) the process produces the polyol ether in at least 30 percent yield based on the amount of the carbonyl compound and the process is characterizable by a molar selectivity ratio of greater than 10:1 for producing the polyol ether over a potential alcohol by-product of formula (IV) R¹R²CHOH (IV), wherein R¹ and R² are as defined previously.

2. The process as in claim 1, the process being characterizable by any one or more of limitations (a) to (k):
(a) the selective hydrogenating conditions comprise a pressure of from 100 kilopascals to 14,000 kilopascals and a temperature of from 24 degrees Celsius to 300 degrees Celsius;
(b) the palladium hydrogenation catalyst is characterized by a catalyst composition of from 0.01 wt % to 30 wt % of palladium and from 0 wt % to 20 wt % of the co-metal based, both based on total weight of the palladium hydrogenation catalyst;
(c) the palladium hydrogenation catalyst is characterized by a catalyst metal weight/weight ratio of from 100 palladium:0 co-metal to 20 palladium:80 co-metal;
(d) the palladium hydrogenation catalyst is characterized by a catalyst loading of from 0.1 wt % to 50 wt % of the palladium hydrogenation catalyst based on weight of the carbonyl compound;
(e) the molar ratio of the polyol to the carbonyl compound is from greater than 5:1 to 30:1;
(f) the process is characterizable by a molar selectivity ratio of greater than 20:1 for producing the polyol ether over a potential alcohol by-product of formula (IV) R¹R²CHOH (IV), wherein R¹ and R² are as defined previously;
(g) the process produces the polyol ether in greater than 70 percent yield within 12 hours of reaction time;
(h) the Brunauer-Emmett-Teller surface area of the acidic mesoporous carbon support is 1000 square meters per gram or greater;
(i) the mesopore surface area of the acidic mesoporous carbon support is 400 square meters per gram or greater;
(j) the process contacts together ingredients consisting essentially of the polyol, carbonyl compound, hydrogen gas, and palladium hydrogenation catalyst; and
(k) the percent mesoporosity is 25% or greater.

3. The process as in claim 2, the process being characterizable by at least each of limitations (a) to (c) and limitation (d).

4. The process as in claim 2, the process being characterizable by at least each of limitations (a) to (c) and limitation (e).

5. The process as in claim 2, the process being characterizable by at least each of limitations (a) to (c) and limitation (f).

6. The process as in claim 2, the process being characterizable by at least each of limitations (a) to (c) and limitation (g).

7. The process as in claim 2, the process being characterizable by at least each of limitations (a) to (c) and limitation (h).

8. The process as in claim 2, the process being characterizable by at least each of limitations (a) to (c) and limitation (i).

9. The process as in claim 2, the process being characterizable by at least each of limitations (a) to (c) and limitation (j).

10. The process as in claim 2, the process being characterizable by at least each of limitations (a) to (c) and limitation (k).

11. The process as in claim 1, wherein one of R¹ and R² is H and the other of R¹ and R² is (C₁-C₅₀)alkyl, (C₂-C₅₀)alkenyl, (C₆-C₁₀)aryl-(C₁-C₅₀)alkyl, (C₆-C₁₀)aryl-(C₂-C₅₀)alkenyl-, or (C₃-C₁₂)cycloalkyl.

12. The process as in claim 1, wherein each one of R¹ and R² independently is (C₁-C₅₀)alkyl, (C₂-C₅₀)alkenyl, (C₆-C₁₀)aryl-(C₁-C₅₀)alkyl, (C₆-C₁₀)aryl-(C₂-C₅₀)alkenyl-, or (C₃-C₁₂)cycloalkyl; or R¹ and R² together with the carbon atom to which they are both attached form a (C₃-C₁₂)cycloalkyl ring.

13. The process as in claim 1, wherein each Q is a covalent bond.

14. The process as in claim 1, wherein each Q independently is (C₁-C₁₄)alkylene or (C₁-C₁₄)heteroalkylene.

15. The process as in claim 1, wherein m is 1.

16. The process as in claim 1, wherein m is from 2 to 100.

17. The process as in claim 1, the process further comprising purifying the polyol ether in such a way so as to separate the polyol ether from at least one of the carbonyl compound, polyol, and any alcohol by-product from a reduction of the carbonyl compound.

18. A process for preparing a palladium hydrogenation catalyst, the process comprising:
impregnating or depositing-adsorbing a PdCl₂ or independently a PdCl₂ and a corresponding co-metal chloride that is lanthanum chloride, yttrium chloride, nickel chloride, zinc chloride, copper chloride, manganese chloride, cobalt chloride, iron chloride, chromium chloride, vanadium chloride, titanium chloride, scandium chloride, or a lanthanoid chloride other than lanthanum chloride on and in an acidic mesoporous carbon support, to give an impregnated or deposited-adsorbed material; and
activatingly reducing the impregnated or deposited-adsorbed material so as to produce a palladium hydrogenation catalyst comprising palladium(0) or a palladium(0)-(co-metal) comprising palladium(0) in the presence of at least one co-metal, wherein the at least one co-metal is lanthanum, yttrium, nickel, zinc, copper, manganese, cobalt, iron, chromium, vanadium, titanium, scandium, or a lanthanoid other than lanthanum; the palladium(0) or palladium(0)-(co-metal) being supported on a surface of the acidic mesoporous carbon support, wherein the acidic mesoporous carbon support is characterizable as having a percent mesoporosity of greater than 15%, wherein percent mesoporosity is equal to 100 times mesopore surface area (square meters per gram) of the acidic mesoporous carbon support divided by Brunauer-Emmett-Teller surface area (square meters per gram) of the acidic mesoporous carbon support; and when a co-metal chloride is employed the depositing-adsorbing steps can be performed sequentially or essentially simultaneously and the activatingly reducing steps can be performed sequentially or essentially simultaneously.

19. A palladium hydrogenation catalyst prepared by the process as in claim 18.
